(19) United States
(12) Patent Application Publication
(10) Pub. No.: US 2007/0280946 A1
(43) Pub. Date: Dec. 6, 2007

(54) Antibody to 5' -Deoxy-5' - Methylthioadenosine And Uses Thereof

(76) Inventors: Yoshito Numata, Osaka (JP); Koji Enomoto, Osaka (JP); Akira Yamauchi, Osaka (JP)

Correspondence Address: John P. White;Cooper & Dunham LLP, 1185 Avenue of the Americas, New York, NY, 10036, US

(21) Appl. No.: 11/547,955

(22) PCT Filed: Apr. 5, 2005
(86) PCT No.: PCT/IL05/00374

§ 371 Date: Jul. 11, 2007

(30) Foreign Application Priority Data
Apr. 5, 2004 (JP) 2004-111373

(51) Int. Cl. A61K 39/395; A61P 19/00; A61P 25/28; A61P 35/00; C07K 16/46; C12N 15/06; C12N 5/16

(52) U.S. Cl. 424/146.1; 435/346; 435/70.21; 530/391.3

(57) ABSTRACT

The present invention provides a new immunogen, a novel antibody having high specificity for MTA (5′-deoxy-5′-methylthioadenosine), methods of treatment and medicaments prepared using said antibody, and sensitive assays for measuring spermidine synthase activity.

This application claims priority of Japanese patent application No 2004-111373 filed 5 Apr. 2004 which is hereby incorporated by reference in its entirety.

Throughout this application various patent and scientific publications are cited. The disclosures for these publications in their entireties are hereby incorporated by reference into this application to more fully describe the state of the art to which this invention pertains.

### TECHNICAL FIELD

The present invention provides an antibody having a high specificity to 5′-deoxy-5′-methylthioadenosine (hereinafter also referred to as MTA), and provides an assay method using the antibody and pharmaceutical uses of the antibody.

### BACKGROUND ART

Spermidine synthase (hereinafter also referred to as SPDS) produces spermidine according to the following reaction scheme. Spermidine is known to be involved in stabilization and conformational change of nucleic acids due to its interaction therewith, to have a facilitatory effect on a variety of nucleic acid synthesis systems and to exhibit a wide range of physiological effects such as activation of protein synthesis system. Therefore, a method for measuring an activity of spermidine synthase in animal tissues has been developed.

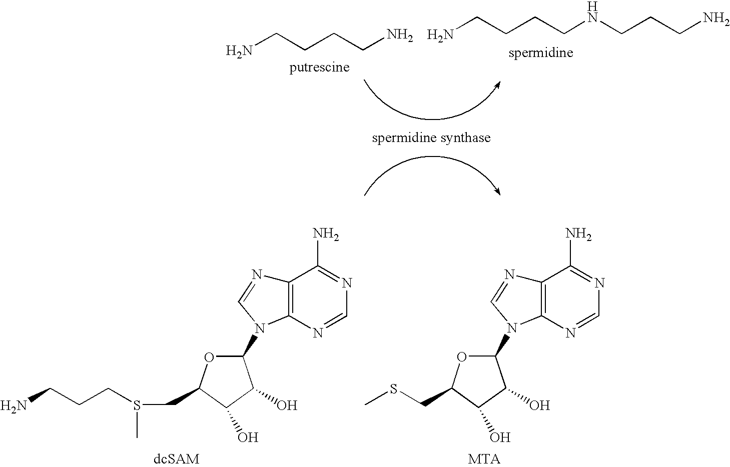

Spermidine is one of three bioactive polyamines, the other two being putrescine and spermine. Polyamines constitute a group of cell components that are important in the regulation of cell proliferation and cell differentiation. Although their exact functions have not yet been clarified, it is assumed that polyamines play an important role in a number of cellular processes such as replication, transcription, and translation.

The polyamine biosynthetic pathway consists of two highly regulated enzymes, ornithine decarboxylase and S-adenosylmethionine decarboxylase, and two constitutively expressed enzymes, spermidine synthase and spermine synthase. Spermidine synthase is a 74 kDa protein that catalyses the 3-aminopropylation of putrescine (1,4-diaminobutane) to produce spermidine. The biosynthesis of spermidine involves decarboxylation of S-adenosylmethionine (SAM) to S-adenosyl-3-methylthiopropanamine (decarboxylated SAM) by SAM decarboxylase, and decarboxylation of ornithine to putrescine by ornithine decarboxylase. Decarboxylated SAM then reacts with spermidine synthase to generate an aminopropylated form of the enzyme, which then transfers the aminopropyl group to putrescine to produce spermidine and 5′-methylthioadenosine (MTA). The active enzyme is a dimer of two identical subunits, requires no cofactors, and uses dcAdoMet as an aminopropyl donor and putrescine as the acceptor.

Putrescine, spermidine and spermine have been found in many living tissues, including cartilage. Their formation, catalyzed by ODC, has been observed during the induction of cartilage transformation in bone. Parathyroid hormone, which stimulates the synthesis of glycosaminoglycans, induces ODC activity and increases polyamine levels in differentiated rabbit costal chondrocytes in culture. Resting cartilage is devoid of putrescine. Ossifying cartilage contains more polyamines than the resting zone (based on tissue weight and DNA content). The amount of spermidine in the ossifying zone is 5-fold higher and that of spermine about 2-fold. The spermidine/spermine ratio is 1.7 in the ossifying cartilage and 0.69 in the resting zone. Only spermidine showed the capacity of displacing proteoglycan subunits from a column of Sepharose 4B-type II collagen (Franco Vittur et al. (1986). A possible role for polyamines in cartilage in the mechanism of calcification. Biochimica et Bioplysica Acta 881:38-45).

The effect of polyamines on the interaction of proteoglycan units with collagen was studied by following the elution of proteoglycans from a column of Sepharose 4B-collagen loaded with proteoglycan subunits. While putrescine and spermine were without effect, spermidine showed a strong capacity in displacing proteoglycan subunits: about 90% of the proteoglycan subunits were removed from the column. Spermine and spermidine increased the activity of alkaline phosphatase produced from cartilage. Spermidine was observed in the cells of the resting zone of preosseous cartilage. Cell staining disappeared, approaching the zone of proliferating and columnar cells. Staining for spermidine is markedly evident in the matrix only at the limit of columnar cells where hypertrophy of chondrocytes initiates. Among the three polyamines, spermidine is the most abundant: a high molar ratio of spermidine/spermine has been taken as an index of rapid growth. The highest amount of spermidine is in the ossifying region.

Polyamine Metabolism

The synthesis of the precursors, putrescine and decarboxylated S-adenosylmethionine (dcAdoMet) is brought about by the action of two decarboxylases ornithine decarboxylase (ODC, EC 4.1.1.17) and S-adenosylmethionine decarboxylase (AdoMetDC, SamDC, EC 4.1.1.51). These enzymes are very highly regulated by means of both growth factors and other stimuli that increase their levels, and by polyamines themselves, which reduce their activity. The combined effect of these agents is to adjust the polyamine levels to that needed for cell growth and development. Alternation in the activities of ODC and AdoMetDC are the major forces in controlling polyamine levels. The activities of the aminopropyltransferases, putrescine aminopropyltransferase (PAPT, spermidine synthase, EC 2.5.1.6) and spermidine aminopropyltransferase (SAPT, spermine synthase, EC2.5.1.22) are controlled primarily through the availability of their substrates. In addition to their de novo synthesis within the cells, polyamines can also be obtained as a result of uptake by a specific transport system. This transport system is regulated both negatively by the intracellular polyamine content and positively by growth factors and oncogenes. The presence of the transport system and its enhanced activity as a result of polyamine depletion is a significant factor in ameliorating the effect of the inhibition of polyamine synthesis. The uptake of exogenous polyamines may be a critical factor in the lack of success in clinical trials of these inhibitors as anti-tumor agents. Finally, polyamine levels can be altered as a result of interconversion, oxidation and efflux. The oxidation of polyamines at the terminal nitrogen atoms is accomplished by Cu⁺²-containing oxidases that appear to be located primarily extracellularly, although their complete absence from the cell has not been established. Interconversion and efflux of polyamines from the cell is facilitated by means of the action of spermidine/spermine-N-acetyltransferase (SSAT, EC 2.3.1.57) which acetylates the aminopropyl end of the polyamines forming N-acetylspermine and N-acetylspermidine. These acetyl derivatives bind less tightly to cellular polyanions and are either excreted or rapidly metabolized. They are oxidized at the internal nitrogen atom by a FAD-dependent oxidase called polyamine oxidase (PAO) splitting of N-acetylaminopropanal and converting spermine into spermidine and spermidine into putrescine. The limiting factor in this pathway is the activity of SSAT, which is normally very low but is induced greatly by an increase in the cellular content of polyamines or by the application of toxic stimuli, which lead to the release of polyamines from membranes and cellular organelles. Under physiological conditions, PAO has little or no activity against non-acetylated polyamines.

As a method for measuring an activity of spermidine synthase, there have been described methods of labeling a substrate, putrescine or decarboxylated S-adenosylmethionine (hereinafter also referred to as dcSAM) with ¹⁴C and measuring the activity by using ion exchange chromatography (J. Biol Chem, 1969, Vol. 244, p 682), paper electrophoresis (Biochem. J, 1978, Vol. 169, p 709) or high performance liquid chromatography (HPLC) (Journal of Chromatography, 1981, Vol. 226, p 208). However, since all of these methods require a step of separating a measured subject after the reaction, these are inefficient and time-consuming.

A method for measuring an activity of SPDS by radioimmunoassay (RIA) has also been described (Journal of Chromatography, 1988, Vol. 440, p 141, J. Biochem. Mol. Biol., 1997, Vol. 30, p 403, and J. Biochem. Mol. Biol., 1997, Vol. 30, p 443). In general, an antibody having a high specificity is required for detection by such immunological methods. In the prior art, antibodies to a reaction byproduct, 5′-deoxy-5′-methylthioadenosine were utilized, but all of these were polyclonal antibodies and had low antibody titers of merely around 2,000-fold. In addition, these also had a low specificity, since they had approximately 1% cross-reactivity with decarboxylated S-adenosylmethionine, which resembles 5′-deoxy-5′-methylthioadenosine in structure. It is difficult to apply such an antibody in an immunological detection method including enzyme linked immunosorbent assay (hereinafter also referred to as ELISA), and thus the detection ability cannot be fully exerted. Therefore, an improvement of such antibodies has been required.

Osteoarthritis (OA) is a common, debilitating, costly, and currently incurable disease. Novel approaches to therapy are clearly required. The disease is characterized by abnormal functioning of chondrocytes, their terminal differentiation and initiation of osteogenesis within articular cartilage tissue, and breakdown of normal cartilage matrix.

Epidemiology of OA

OA, also erroneously called degenerative joint disease, represents failure of a diarthrodial (movable, synovial-lined) joint. In idiopathic (primary) OA, the most common form of the disease, no predisposing factor is apparent.

Secondary OA is pathologically indistinguishable from idiopathic OA but is attributable to an underlying cause. OA is the most common of all human joint disorders and is the most prevalent arthritic condition in the United States and around the world. Estimates of OA prevalence based on clinical evaluation in various studies show that more than 90% of the population over the age of 70 has OA. The invention is aimed at novel avenues of therapy and prevention of the disease.

Pathogenesis of OA

OA is a heterogeneous group of conditions that lead to joint symptoms and signs associated with defective integrity of articular cartilage, in addition to related changes in the underlying bone at the joint margins. OA may be either idiopathic (i.e., primary) or secondary to other medical conditions (inflammatory, biochemical, endocrine-related, metabolic, and anatomic or developmental abnormalities). Age is the most powerful risk factor for OA but major trauma and repetitive joint use are also important risk factors for OA. The pattern of joint involvement in OA is also influenced by prior vocational or avocational overload.

The disease has two general stages: (1) compensated and (2) decompensated. Currently, most investigators feel that the primary changes occur in cartilage extracellular matrix due to exogenous reasons (i.e., load, injury etc.). Then, a defect in the collagen network of the cartilage is apparent, and lysosomal enzymes and secreted proteases (MMPs, plasmin, cathepsins) probably account for the observed initial alterations in cartilage matrix. Their synthesis and secretion are stimulated by IL-1 or by other factors (e.g., mechanical stimuli). In the initial stage of disease, compensatory cellular response is activated. Secreted by chondrocytes, protease inhibitors like TIMP and PAI-1 work to stabilize the system by opposing the protease activity. Growth factors such as IGF-1 and TGF-β are implicated in repair processes that may heal the lesion or, at least, stabilize the process by activating proliferation of cells of chondrogenic lineage. Finally, this leads to the accumulation of hypertrophic chondrocytes. The latter cells have marked biosynthetic activity that is expressed in increasing the proteoglycan (PG) concentration, associated with thickening of the cartilage (“compensated” OA). The compensatory mechanisms may maintain the joint in a reasonably functional state for years. However, the repair tissue does not hold up and the rate of PG synthesis falls off with full-thickness loss of cartilage. This marks the decompensated stage of OA. Following the destruction of the articular cartilage, there is migration of progenitor cells to the sites of tissue damage. These cells proliferate and differentiate into four cell types: osteoblasts, chondroblasts, chondroclasts and fibroblasts, which combine to form bony structures called osteophytes which protrude into the joint space, thus inhibiting its movement. Finally, gradual replacement of cartilage with bone occurs.

The reason for this phenomenon is unknown. One possibility is that in OA, the normal inhibitory growth control of articular chondrocytes or synovial membrane fibroblasts is altered. This enables accumulation of two types of cells that cannot be found in normal articular cartilage: (1) immature mesenchymal and bone marrow cells with modified properties, and (2) hypertrophic articular chondrocytes. Previous results have clearly shown that hypertrophic chondrocytes may trigger osteogenesis by secretion of angiogenic and osteogenic factors. (Horner, A., Bishop, N. J., Bord S., Beeton, C., Kelsall, A. W., Coleman, N. and Compston, J. E. (1999). Immunolocalisation of vascular endothelial growth factor (VEGF) in human neonatal growth plate cartilage. J. Anat. 194: 519-524).

In OA, therapeutic interference may target three main processes:

inhibition of initial cartilage damage—one of the accepted therapeutic strategies, combining recommendations to reduce the physical pressure on the joint and treatment with inhibitors of metalloproteinases;

inhibition or attenuation of total cartilage destruction at later stages—implies the therapeutic activation of processes connected to cartilage rehabilitation, namely, the promotion of proper differentiation of mesenchymal progenitors into mature chondrocytes capable of producing fully functional articular cartilage tissue;

inhibition or attenuation of osteophyte formation at the end stage of the disease—implies the therapeutic inhibition of ectopic osteogenesis at the site of articular cartilage.

Changes in gene expression caused by IL-1, FGF-2 and mechanical stress, which are known osteogenic factors, may be connected to OA development and, therefore, should be opposed by therapeutic intervention. The Assignees of the present invention have previously found that one of the genes that is upregulated by FGF-2 is the spermidine synthase gene (see co-assigned U.S. Pat. No. 6,696,454, which is hereby incorporated by reference in its entirety). This implicates involvement of the spermidine synthase gene in the OA pathway.

### DETAILED DESCRIPTION OF THE INVENTION

An object of the present invention is to provide a polyclonal antibody and a monoclonal antibody binding specifically to MTA, and a sensitive assay method utilizing said antibodies.

An antibody has conventionally been prepared with an immunogen in which a sugar moiety of MTA undergoes ring-opening and binding of carrier protein by periodate oxidation (Erlanger et al., 1964), represented by the general formula (IV): 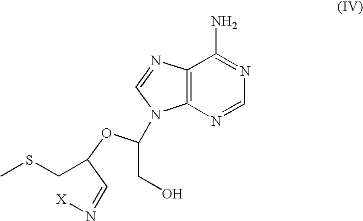
wherein, X is a carrier protein.

However, as a result of extensive investigations, the present inventors have found that an antibody specifically recognizing MTA can be prepared by using an immunogen in which a linker is introduced to a base moiety of MTA, represented by the general formula (III): 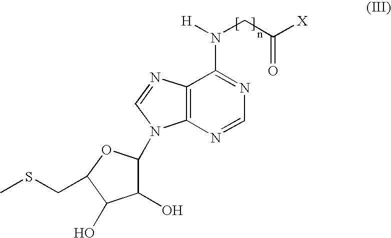
wherein n is an integer of 1 or more, and X is a carrier protein.

That is, the present invention comprises the following:

(1) a monoclonal antibody binding specifically to 5′-deoxy-5′-methylthioadenosine;

(2) a monoclonal antibody according to (1) having not more than 0.1% of cross-reactivity to decarboxylated S-adenosylmethionine, based on the reactivity to 5′-deoxy-5′-methylthioadenosine taken as 100%;

(3) a monoclonal antibody according to (1), wherein the crossreactivity is not more than 0.01%;

(4) a polyclonal antibody having 0.1% or less of cross-reactivity to decarboxylated S-adenosylmethionine, as compared to the reactivity to 5′-deoxy-5′-methylthioadenosine taken as 100%;

(5) a compound represented by the general formula (I): 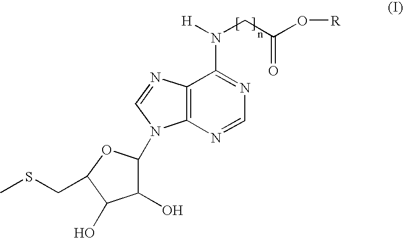
wherein n is an integer of 1 or more 1, and —C(═O)—OR is an active ester;

(6) the compound according to the above (5), wherein n in the compound represented by the general formula (I) is 5;

(7) the compound according to the above (6), wherein R is represented by the following formula (II):

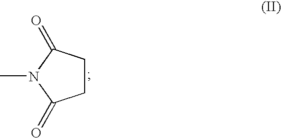

(8) an immunogen, wherein a carrier protein is bound to the purine group of 5′-deoxy-5′-methylthioadenosine;

(9) the immunogen according to the above (8) represented by the general formula (III): 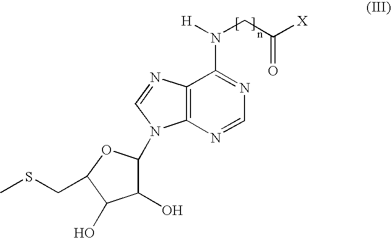
wherein n is an integer of 1 or more, and X is a carrier protein;

(10) the immunogen according to the above (9), wherein n in the compound represented by the general formula (III) is 5;

(11) the immunogen according to the above (10), wherein X is keyhole limpet hemocyanin;

(12) the polyclonal antibody according to the above (4), which is directed against the immunogen described in any one of the above (8) to (10);

(13) a monoclonal antibody according to any one of the above (1) to (3), which is directed against the immunogen described in any one of the above (8) to (10);

(14) a hybridoma, which produces a monoclonal antibody binding specifically to 5′-deoxy-5′-methylthioadenosine;

(15) the hybridoma according to the above (14), which is deposited under the accession number FERM-ABP10226;

(16) the monoclonal antibody according to any one of the above (1) to (3), which is produced by the hybridoma described in the above (15);

(17) the antibody according to any one of the above (1) to (4), which is labeled;

(18) the antibody according to the above (17), wherein the label is an enzyme, a fluorescent substance, a colored bead, a radioisotope, a metal or biotin;

(19) the antibody according to the above (18), wherein the label is peroxidase;

(20) the antibody according to the above (18), wherein the label is europium cryptate;

(21) an assay method using the antibody described in any one of the above (1) to (4);

(22) the assay method according to the above (21), wherein the assay method using an antibody is ELISA;

(23) the assay method according to the above (21), wherein the assay method using an antibody is a homogeneous assay;

(24) the assay method according to the above (23), wherein the homogeneous assay is an assay utilizing fluorescence resonance energy transfer;

(25) the assay method according to any one of the above (21) to (24), which comprises measuring an activity of spermidine synthase; and

(26) a method according to any one of the above (21) to (25), which comprises screening a therapeutic/prophylactic agent for osteoarthrosis.

By using an antibody of the present invention, 5′-deoxy-5′-methylthioadenosine can be detected in high sensitivity, and a rapid, simple and sensitive assay such as ELISA and a homogeneous assay has been made possible. Moreover, the antibody of the present invention can be used in measuring an activity of spermidine synthase and further in screening a therapeutic/prophylactic agent for Osteoarthritis.

In an additional aspect, the present invention relates to a method for the treatment of a subject in need of treatment for a disease, this method comprising administering to said subject an amount of an inhibitor of MTA sufficient to effect a substantial inhibition of MTA so as to thereby treat the subject.

The disease involves spermidine synthase, and is preferably osteoarthritis but may also be a proliferative disease including cancer, osteoporosis, Alzheimer's disease, and hypertension. Treatment of any disease involving pathological activity of spermidine synthase or any other enzyme in the spermidine biosynthesis pathway is contemplated.

The inhibitor is preferably any one of the antibodies disclosed herein, such as in items 1-4, 12-13 and 16-20 above, optionally conjugated to a carrier, compositions comprising said antibodies and/or compounds comprising said antibodies, such as items 5-7 above, all as detailed herein. This aspect of the present invention further provides for a pharmaceutical composition comprising any one of the antibodies or compounds disclosed herein.

The antibodies and compounds disclosed herein are essentially inhibitors of the polyamine biosynthetic pathway, and as such, may be used in the treatment of OA in a mammalian subject, alone or as a component of a pharmaceutical composition optionally containing additional active ingredients.

Any of the antibodies, compounds and compositions disclosed herein may be used in therapeutic compositions, preferably for the treatment of OA. According to a preferred embodiment, the composition of the invention may optionally further comprise a pharmaceutically or veterinarily acceptable carrier, excipient and/or diluent.

Animal Models

In addition to the various model systems disclosed herein, the pharmaceutical efficacy of the compound of the present invention can be evaluated according to the following test systems: in vitro cell culture, ex vivo cell culture, ex vivo organ culture and in vivo animal model. Said systems may express MTA or spermidine synthase endogenously or exogenously. This test system may optionally further comprise endogenous and/or exogenous agents that provide suitable conditions for the expression of spermidine or MTA and for the detection of an end-point indication for determining any one of chondrocyte proliferation, chondrocyte final differentiation, angiogenesis and osteoclastogenesis.

Depending on the test assay system chosen, inhibition of chondrocyte proliferation, chondrocyte final differentiation, angiogenesis and osteoclastogenesis can be observed in a variety of ways, including intracellular staining assays (including immunohistochemical) and assays affecting an observable parameter; e.g., a physiological readout, such as change in cell cycle.

According to a preferred embodiment, the test system used by the method of the invention for evaluating the effect of said candidate inhibitor is an in vitro transfected cell culture. The cells employed carry an exogenously expressed spermidine synthase.

In an alternative embodiment, the test system used by the method of the invention for evaluation purposes is an ex vivo bone culture, comprising endogenously expressed spermidine synthase. Preferably, the bone culture used is an embryonic bone culture.

Another alternative test system may be an in vivo system, which is an animal model system. According to the method of the invention, use of an animal model for evaluation purposes enables utilizing the development of arthritis as an end-point indication. Where used as an end-point indication, development of arthritis may be determined, for example, by measuring paw thickness of said animal. Any increase in the size of the paw that is less than the increase observed in a control is indicative of inhibition of chondrocyte proliferation, chondrocyte final differentiation, angiogenesis and osteoclastogenesis, or development of arthritis by the test candidate inhibitor.

In one preferred test system, an appropriate animal model may be a transgenic mouse.

In yet another preferred in vivo test system, an arthritic mammalian model expressing endogenous spermidine synthase may be used by the evaluating method of the invention. According to this embodiment, the arthritic animal enables utilizing the development of arthritis as an end-point indication.

According to a particularly preferred embodiment, the arthritic mammal may be an arthritic rat or an arthritic mouse.

Another alternative test system may be an in vivo system, which is an animal model. According to the method of the invention, use of an animal model for evaluation purpose, enables utilizing development of arthritis as an end-point indication. In cases of use as an end point indication, development of arthritis may be determined by measuring paw thickness of the test animal. Any increase of the thickness of the paw less than that of a control is indicative of inhibition of chondrocyte proliferation, chondrocyte final differentiation, angiogenesis and osteoclastogenesis, or development of arthritis by the test candidate inhibitor.

In one preferred test system, an appropriate animal model may be a transgenic mouse expressing exogenous spermidine synthase. More particularly, the transgenic mouse expresses the spermidine synthase gene under the collagen type II promoter.

According to this particular embodiment, evaluation of the effect of a candidate spermidine synthase inhibitor using this in vivo test system, involves applying the test candidate inhibitor to said transgenic mouse under conditions which normally lead to biosynthesis of spermidine. These particular suitable conditions may be, for example, providing a spermidine synthase substrate such as SAM to the tested transgenic mice prior to application of the candidate inhibitor.

In yet another preferred in vivo test system, an arthritic mammalian model expressing endogenous spermidine synthase may be used by the evaluating method of the invention. According to this embodiment, the arthritic animal enables utilizing the development of arthritis as an end-point indication. Development of arthritis may be determined by measuring the paw thickness of an examined arthritic mammal. Less increase in the size of the paw, as compared to a control, is indicative of inhibition of any one of chondrocyte proliferation, chondrocyte final differentiation, angiogenesis and osteoclastogenesis, and development of arthritis by said test candidate inhibitor. Accordingly, the effect of the candidate inhibitor on the arthritic animal test system will be further examined using the differentiation and proliferation end-points as discussed herein above.

### BRIEF DESCRIPTION OF THE DRAWINGS

FIG. 1. This figure sets forth a graph showing reactivity of the monoclonal antibody MTA-7H8 of the present invention to MTA and to precursor dcSAM in ELISA;

FIG. 2. This figure sets forth graphs showing an activity of SPDS measured by ELISA of the present invention. Graph A shows a standard curve; graph B shows measuring values of ELISA obtained at respective amounts of enzyme, which is converted into a binding rate of labeled MTA; graph C shows production amounts of MTA calculated based on the results of A and B at respective amounts of enzyme;

FIG. 3. This figure sets forth a graph showing an inhibition activity of 4-methylcyclohexylamine to SPDS measured by ELISA of the present invention;

FIG. 4. This figure sets forth a graph showing reactivity of the monoclonal antibody MTA-7H8 of the present invention to MTA and to precursor dcSAM in HTRF;

FIG. 5. This figure sets forth graphs showing an activity of SPDS measured by HTRF of the present invention. Graph A shows a standard curve of MTA; graph B shows measuring values of HTRF obtained at respective amounts of enzyme, which is converted into a binding rate of labeled MTA; graph C shows production amounts of MTA calculated based on the results of A and B at respective amounts of enzyme;

FIG. 6. This figure sets forth a graph showing an inhibition activity of 4-methylcyclohexylamine to SPDS measured by HTRF of the present invention; and

FIG. 7. This figure sets forth a graph showing reactivity of the monoclonal antibody MTA-7H8 of the present invention to MTA and to precursor dcSAM in SPA.

FIG. 8. This figure sets forth a graph showing the correlation between MTA levels determined by the present invention and spermidine levels measured by an HPLC method.

### PREFERRED EMBODIMENTS OF THE PRESENT INVENTION

“5′-deoxy-5′-methylthioadenosine” is produced from decarboxylated S-adenosylmethionine and the like during a biosynthesis process of polyamines such as spermidine and spermine. 5′-deoxy-5′-methylthioadenosine is used not only in measuring an activity of polyamine synthase in each tissue, but also as a tumor marker.

“Decarboxylated S-adenosylmethionine” serves as an aminopropyl group donor in the synthesis of spermidine, spermine and the like, which is used as a substrate of spermidine synthase or spermine synthase in a polyamine biosynthesis process. Decarboxylated S-adenosylmethionine is produced from S-adenosylmethionine (SAM) by decarboxylation reaction with decarboxylation enzyme.

The term “cross-reactivity” means an immuno-crossreactivity. When an antibody obtained by immunization with a certain antigen exhibits a binding reaction with another antigen (a related antigen), this reaction is a cross-reaction. A degree of reaction amount of the antibody with a related antigen can be expressed as cross-reactivity based on a reaction amount of the antibody with a desired antigen. In the present specification, cross-reactivity is expressed as relative value (%) of affinities to MTA to dcSAM. The lower the value is, the lower the cross-reactivity is, and therefore the antibody shows it has specificity to a desired antigen. A cross-reaction often occurs mainly due to the fact that a desired antigen structure and a related antigen structure are similar to each other.

It is considered that cross-reaction of the antibody of the present invention occurs between 5′-deoxy-5′-methylthioadenosine and decarboxylated S-adenosylmethionine due to their similarity in structure. One embodiment of such measuring method is described in the Examples. The polyclonal antibody according to the present invention is a polyclonal antibody having not more than 0.1% cross-reactivity to decarboxylated S-adenosylmethionine, based on the reactivity to 5′-deoxy-5′-methylthioadenosine taken as 100%. The monoclonal antibody according to the present invention is a monoclonal antibody binding specifically to 5′-deoxy-5′-methylthioadenosine, and preferably a monoclonal antibody having not more than 0.1% cross-reactivity to decarboxylated S-adenosylmethionine, more preferably having not more than 0.01% cross-reactivity, based on the reactivity to 5′-deoxy-5′-methylthioadenosine taken as 100%.

The term “antibody titer” means an amount of antibody binding to an antigen contained in a unit volume of an antiserum in a serum reaction. Practical measurement is carried out by adding a determined amount of antigen to a dilution series of antiserum. A measurement value is represented by the maximum dilution rate where the reaction can occur.

The term “affinity” means an associative strength between an antigen and an antibody. In the present description, affinity is represented in terms of a concentration index of MTA or dcSAM at which 50% of association amount of labeled MTA to an antibody is inhibited (IC50 value). IC50 value is calculated by using a logistic curve regression model (Rodbard et al., Synposium on RIA and related procedures in medicine, P165, Int. Atomic Energy Agency, 1974).

“Hapten” is a partial or incomplete antigen. Hapten is a substance mainly of low molecular weight, which is not by itself capable of stimulating production of an antibody. An antibody directed to hapten can be obtained by immunizing with an artificial antigen in which hapten is linked to a carrier protein by a chemical method or a crosslinker. In the present invention, it is considered difficult for 5′-deoxy-5′-methylthioadenosine to produce an antibody by itself, because it is a low molecular compound, and thus a complex of 5′-deoxy-5′-methylthioadenosine with a carrier protein which is usually a heterologous protein and a synthetic polypeptide is prepared and used as an immunogen.

When binding a hapten to a carrier protein, usually a derivative of hapten introduced with a reactive functional group is prepared. The derivative is referred to as “activated hapten” in the present description. An activated hapten may have a linker of suitable length for producing an antibody having high specificity. When introducing a reactive functional group, a recognition site in the hapten, which is a partial structure recognized by an antibody, is affected depending on an introduction position of the reactive functional group. Therefore, it is important to select appropriately an introduction position of the reactive functional group according to a desired antibody.

“Activated hapten” according to the present invention is not a conventional compound having a sugar moiety of increased reactivity in nucleotides, but a compound characterized by being introduced with a reactive functional group at a purine group of the base moiety, preferably a compound introduced with a reactive functional group at NH group of 6-position represented by the general formula (I): 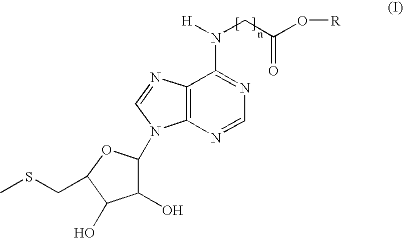
wherein n is an integer not less than 1, and —C(═O)—OR is an active ester.

A linker which links a reactive functional group to hapten can have any length, including, for example, as a linear alkylene chain, preferably a chain having a length of not less than one carbon atom, more preferably having a length of one to ten carbon atoms, and especially preferably having a length of five carbon atoms. Any reactive functional group can be used as long as it is highly reactive and binds to an amino residue of a carrier protein, preferably being in the form of an active ester (for example, succinimido ester, phthalimido ester and maleimido ester). An activated hapten is especially preferably a compound represented by the formula (V):

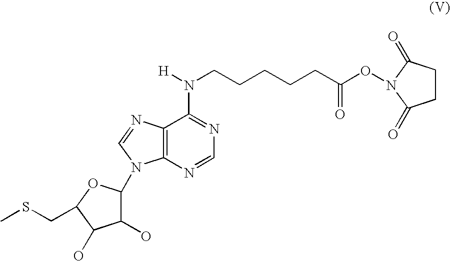

As a “carrier protein”, any type of proteins known to increase an antigenicity can be used. Examples of the carrier protein include a macromolecule such as bovine serum albumin (BSA), bovine Thyroglobulin (BTG) and keyhole limpet hemocyanin (KLH), and a synthetic polypeptide and the like.

The term “immunogen” as used herein refers to a substance capable of generating or inducing an immune response in an organism. The immunogen of the present invention comprises an activated hapten having a partial structure which may be an antigenic determinant of 5′-deoxy-5′-methylthioadenosine and a carrier protein linked to the hapten, and is preferably an immunogen in which a carrier protein is linked to a purine group in 5′-deoxy-5′-methylthioadenosine, more preferably an immunogen represented by the general formula (III): 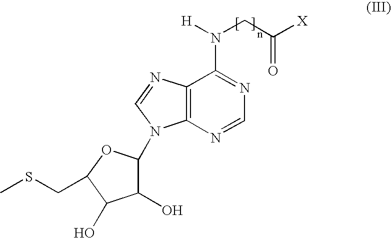
wherein n is an integer not less than 1, and X is a carrier protein, still more preferably an immunogen in which n is 1 to 10, and especially preferably an immunogen in which n is 5. Preferably, X is a keyhole limpet hemocyanin.

An immunogen used for producing the antibody of the present invention can be prepared by using an activated hapten and a carrier protein, according to the active ester method described in Antibodies: A Laboratory Manual, (1989) (Cold Spring Harbor Laboratory Press) and the like. Other methods described in Antibodies: A Laboratory Manual, (1989) (Cold Spring Harbor Laboratory Press) and the like, for example carbodiimide method, glutaraldehyde method and diazo method may also be used to preparing the immunogen.

For example, a polyclonal antibody is obtained by collecting blood from a mammal immunized with an immunogen. In the said methods, as the mammal to be immunized with the immunogen, rabbit, goat, sheep, mouse, rat and the like are generally used. In the present invention, to compare the efficiency of a monoclonal antibody and a polyclonal antibody at the same species, mouse was used as an immunized animal.

The immunizing method can be carried out according to a usual method such as by administering an immunogen to a mammal via intravenous, intradermal, subcutaneous or intraperitoneal injection or the like. More specifically, an immunogen is, for example, diluted into an appropriate concentration with phosphate buffered saline (PBS), saline and the like, and administered to a test animal in multiple doses at a two- to three-week interval in combination with a usual adjuvant according to need. When using a mouse, a dosage is 50 to 100 μg per mouse. “Adjuvant” refers to a substance which nonspecifically enhances an immune response to an antigen when administered together with the antigen. Examples of an adjuvant usually used include whooping-cough vaccine, Freund's adjuvant and the like. By collecting blood from a mammal three to ten days after the last immunization, an antiserum can be obtained. The antiserum may be used as it is or as a polyclonal antibody after purification.

Examples of a method of purifying a polyclonal antibody include non-specific purification method and specific purification method. The non-specific purification method is directed to obtain mainly an immunoglobulin fraction by salting out, ion exchange chromatography and the like. Examples of the specific purification method include affinity chromatography with an immobilized antigen and the like.

A method for producing a monoclonal antibody is conducted by preparing fusion cells (hybridoma) of plasma cells (immunocyte) of an immunized mammal with an immunogen with plasmacytoma cells (myeloma cells) of the mammal, selecting clones which produce a desired monoclonal antibody recognizing 5′-deoxy-5′-methylthioadenosine, and cultivating the clones. The monoclonal antibody can be produced essentially according to the standard methods (see, Kohler, G. and Milstein, C., Nature, 256, 495-497, (1975)).

In the above method, the immunized mammal with the immunogen is preferably selected in view of the compatibility with plasmacytoma cells used in cell fusion, and mouse and rat may be used for such purpose. The immunizing method is similar to the preparation of a polyclonal antibody, except that spleen cells are collected from an immunized mammal three to ten days after the last immunization.

A hybridoma can be obtained from the resultant immunocyte according to, for example, the method described in “Experimental Manual for Molecular Cell Biology” (Takeichi Horie et al., 1994, Nankodo), with the aim of producing cells which can be subcultured, by fusing the immunocyte producing an antibody with plasmacytoma cells in the presence of Sendai virus or polyethylene glycol. Plasmacytoma used in the method is preferably derived from the same homothermal animal species among homothermal animals. For example, when fusing with spleen cells obtained from an immunized mouse, mouse myeloma cells are preferably used. As plasmacytoma cells, known cells such as p3×63-Ag8.UI may be used.

A hybridoma producing a desired antibody can be obtained by selecting with a HAT medium (hypoxanthine-, aminopterin- and thymidine-added medium) and screening for the binding of an antibody secreted in a culture supernatant to an antigen in the stage of a colony being ascertained.

A method for screening, which is exemplified by a variety of methods generally used for detecting an antibody, such as spotting, agglutination reaction, western blotting and ELISA, is preferably conducted according to ELISA utilizing reactivity to 5′-deoxy-5′-methylthioadenosine as an indicator for a culture supernatant of hybridoma as described in the below Examples. By the method of screening, a cell line producing a desired antibody which reacts specifically to 5′-deoxy-5′-methylthioadenosine can be screened.

Cloning of the cell line producing a desired antibody obtained by screening can be conducted according to usual limiting dilution, soft agar method and the like. The cloned hybridoma may be cultivated in a large scale in serum-added medium or serum-free medium according to need. According to the cultivation, a desired antibody of relatively high purity can be obtained as a culture supernatant. The desired antibody can be recovered abundantly as murine ascites by inoculating a hybridoma intraperitoneally into a mammal such as mouse having compatibility to the hybridoma.

As a specific example of the hybridoma, there is exemplified the hybridoma (a clone of MTA-7H8) obtained according to the conventional methods, that is a hybridoma prepared from an immunogen comprising 6-[5-(2,5-dioxopyrrolidin-1-yloxy)pentyl]-5′-deoxy-5′-methylthioadenosine described in the Example below and KLH. The hybridoma (“Mouse hybridoma MTA-7H8”) was deposited at the International Depositary Authority, and received the International depositary accession number FERM-ABP10226. (Said hybridoma was also previously deposited at the International Patent Organism Depositary in National Institute of Advanced Industrial Science and Technology (Tsukuba Central 6, 1-1-1 Higashi, Tsukuba, Ibaraki, Japan) on Mar. 2, 2004, under the accession number FERMP-p19707). A culture supernatant and murine ascites containing the hybridoma which produces the antibody of the present invention may be used as a crude antibody solution without purification or modification. Alternatively, these may be purified by conventional methods such as ammonium sulfate fractionation, salt precipitation, gel filtration, ion exchange chromatography, affinity chromatography and the like to result in a purified antibody.

The antibody of the present invention possesses the following physicochemical and immunological properties:

(1) specificity: cross-reactivity to decarboxylated S-adenosylmethionine is less than 0.1%, preferably less than 0.01%.

(2) immunoglobulin class: it belongs to IgG1 (k).

(3) molecular weight: heavy chain 50 kDa, light chain 27 kDa.

Upon assaying with an antibody as described above, the antibody by itself may usually be labeled with various agents to allow its activity to be detected. An antibody can be labeled by conventional methods such as described in “Experimental Manual for Molecular Cell Biology” (Takeichi Horie et al., 1994, Nankodo). Examples of the various agents include a chemiluminescent substance, an enzyme, a fluorescent substance, colored beads, a radioisotope, an element, a metal, biotin and the like. Specific examples include the following, but are not limited thereto. The chemiluminescent substance refers to, for example, luminol and acridinium ester. The enzyme refers to, for example, β-galactosidase, alkaline phosphatase and peroxidase. The fluorescent substance refers to, for example, europium cryptate, FITC (fluorescein isothiocyanate) and RITC (tetramethylrhodamine isothiocyanate). The colored beads refer to, for example, protein A beads, wheat germ agglutinin (WGA) beads and streptavidin beads. The radioisotope refers to, for example, ¹⁴C, ¹²⁵I, and ³H. The element refers to, for example, a lanthanide element such as europium. The metal refers to, for example, ferritin and colloidal gold. An especially preferred label in the present invention is europium cryptate.

Europium cryptate is a fluorescent substance formed by the inclusion of a europium ion of rare earth element into the intramolecular central cavity of a cyclic trisbipyridine ligand (cryptate). A characteristic “cage” structure of cryptate shields an europium ion from water molecules and transfers an excitation light energy absorbed by the cryptate to the europium ion.

The present invention includes an assay method using an antibody directed to 5′-deoxy-5′-methylthioadenosine which may be labeled as described above or may not. The assay method using an antibody may be a competitive measurement or noncompetitive measurement, and may be a homogeneous assay (measurement in homogeneous system) or a heterogeneous assay (measurement in heterogeneous system). Specifically, the assay may be conducted according to the conventional methods such as enzyme immunoassay (EIA), enzyme linked immunosorbent assay (ELISA), fluoroimmunoassay (FIA), radioimmunoassay (RIA), time-resolved fluoroimmunoassay (TR-FIA), chemiluminescent immunoassay, immunoblotting, western blotting and immunostaining. The assay includes a drug screening in order to develop a therapeutic/prophylactic agent, and also includes an assay concerning to diagnosis of diseases.

A preferable specific method of the assay using the antibody of the present invention includes ELISA. ELISA is a method utilizing an antibody or antigen labeled with an enzyme to quantify an amount of antibody or antigen by an activity of a label-enzyme. To separate an antigen-antibody binding reactant from a labeled antigen and an antibody in free form, immobilized antibody and antigen are used. For immobilization, agarose, the inside of a microtiter plate, latex particle and the like may be used. Specific examples of ELISA include a competitive immunoassay, a double antibody sandwich immunoassay and the like. Examples of the labeled enzyme include a horseradish peroxidase (hereinafter also referred to as HRP), alkaline phosphatase and the like. Preferred is horseradish peroxidase.

An additional preferable specific method of the assay using the antibody of the present invention is a homogeneous assay. The homogeneous assay refers to a measurement method which is conducted in solution (homogeneous system) from start to finish. It is an assay method capable of providing data with high accuracy, with the added advantage of requiring no washing step. It may also be easily miniaturized as it does not require a solid phase, and thus may be applied to high-throughput screening which affords saving of expensive reagents and cutting of cost and energy. Typical examples of the assay include SPA (Scintillation Proximity Assay), fluorescence polarization assay (FP), an assay using fluorescence resonance energy transfer (hereinafter also referred to as FRET) and the like.

A preferred example of the homogeneous assay is an assay method using FRET. FRET is a technique utilizing an energy transfer between two fluorescent substances, called a donor and an acceptor.

Among assays using FRET, a preferred embodiment is Homogeneous Time-Resolved Fluorescence (hereinafter also referred to as HTRF®). HTRF is a measurement method based on FRET, which uses two fluorescent label substances consisting of europium cryptate and XL665 (allophycocyanin derivative, which is a fluorescent protein originated from blue-green algae). In HTRF, measurement is conducted based on the principle that when irradiating europium cryptate with excitation light at 337 nm, europium cryptate emits long-lived fluorescence at 620 nm, but if there is XL665 adjacent to europium cryptate due to the formation of immunoconjugate and the like, fluorescence resonance energy transfer takes place between europium cryptate and XL665, and XL665 receives the energy and emits long-lived fluorescence at 665 nm. Combination of measurements of fluorescence at two wavelengths (620 nm and 665 nm) allows a complete homogenous measurement less subjected to color quenching (inner filter effect) and diminishes background fluorescence which can result in false positive identification.

Another preferred example of the homogeneous assay is the SPA method®. The SPA method uses an antibody linked to a SPA bead and a RI-labeled antigen. When the antibody binds to the antigen, the SPA bead also comes close to the antigen, and thus a β ray radiated from the antigen reaches a scintillator in the SPA bead resulting in a measurable emission. As a non-binding antigen does not affect to the measurement, it is possible to perform the measurement without removing an antigen not binding to an antibody.

Spermidine synthase (SPDS) catalyses a reaction of converting decarboxylated S-adenosylmethionine to spermidine by transferring an aminopropyl group in decarboxylated S-adenosylmethionine to putrescine. In the reaction, 5′-deoxy-5′-methylthioadenosine is produced as a byproduct. Therefore, the assay method of the present invention is able to detect 5′-deoxy-5′-methylthioadenosine specifically and sensitively and is useful for an activity measurement of spermidine synthase.

Further, the assay method of the present invention can be used in a screening of subject substances such as those inhibiting an activity of spermidine synthase. In such screening, a subject substance is examined as to whether it inhibits binding of a recombinant spermidine synthase (purified or partially purified) prepared by an expression vector and the like to a enzyme substrate (e.g., dcSAM and putrescine) when the enzyme is maintained under conditions enabling the enzyme to bind to the substrate (e.g., 0.1 m phosphate buffer, pH 7.4, at room temperature) in the presence of the subject substance, in other words, the subject substance is evaluated on the production amount of 5′-deoxy-5′-methylthioadenosine which is a byproduct. In the screening, the subject substance may be selected from peptides, proteins, non-peptidic compounds, synthetic compounds (such as a low molecular weight compound), fermented products, cell extracts, plant extracts, animal tissue extracts and the like, or may be a sample containing these substances.

A candidate substance can be selected by the screening method according to which a subject substance is selected as a candidate substance when a detected value of 5′-deoxy-5′-methylthioadenosine in a well of an assay plate to which the subject substance is added differs from a detected value of 5′-deoxy-5′-methylthioadenosine in a well of an assay plate to which the subject substance is not added. For example, for a degree of decrease/increase of a detected value of 5′-deoxy-5′-methylthioadenosine, when a detected value of 5′-deoxy-5′-methylthioadenosine in a well added with a subject substance is increased or decreased by 10%, preferably 30%, and especially preferably not less than 50% compared to a detected value of 5′-deoxy-5′-methylthioadenosine in a well without adding the subject substance, the subject substance can be selected as a substance candidate.

The candidate substance selected by the screening is a potential therapeutic/prophylactic agent for diseases known to involve spermidine synthase, such as osteoarthrosis, proliferative diseases including cancer, osteoporosis, Alzheimer's disease, and hypertension. In a preferred embodiment, the assay method of the present invention is utilized for searching a therapeutic/prophylactic agent for osteoarthrosis.

Osteoarthrosis (OA) is a joint disease accompanied with a chronic arthritis, which causes cartilage destruction and change of bone and cartilage by degeneration of a join component. There are primary and secondary osteoarthrosis caused by trauma and disease. OA attacks a loaded joint and is common in old age. Recently, a genetic profiling of a human articular cartilage progenitor cells (HMSC) has been conducted, and it has been described that basic fibroblast growth factor 2 (bFGF-2), which is an osteogenic factor, activates transcription of the gene coding for spermidine synthase. This indicates that a polyamine biosynthetic pathway, especially spermidine biosynthesis, more specifically a spermidine synthase is potentially involved in osteoarthrosis (hereinafter also referred to as OA). It is thus expected that the reaction control of spermidine synthase is helpful in treating/preventing OA diseases, and it is further thought that an inhibitor of the enzyme is useful as a therapeutic/prophylactic agent for the diseases described above (WO 02/058623).

As detailed hereinabove, an additional aspect of the present invention relates to a method for the treatment of a subject in need of treatment for OA, this method comprising administering to said subject an amount of an inhibitor of spermidine biosynthesis effective to inhibit a substantial inhibition of spermidine biosynthesis so as to thereby treat the subject.

By “treatment” is meant the alleviation of the disease state and alleviation of the progression thereof, including the partial or full relief of symptoms associated with the specified disease. “Treatment” may also prevent the disease or delay its onset.

In a preferred embodiment, the method of the invention is intended for treating a mammalian subject, preferably, a human. Therefore, by “patient”, “mammalian subject” or “subject in need” is meant any mammal for which the therapy is desired, including human, bovine, equine, canine, and feline subjects, preferably a human patient.

The therapeutic method of the invention comprises administering a therapeutically effective dose of said inhibitor to a subject in need. As used herein, “therapeutically effective dose” means a dose necessary to achieve a selected result. For example, a therapeutically effective dose of the inhibitor or the composition of the invention is a dose effective for the treatment of the osteoarthritic pathology. This will be expanded in the pharmacology and drug delivery section.

The present invention further provides a therapeutic composition for the treatment of OA. This composition of the invention comprises as an active ingredient an inhibitor of one or more steps in the polyamine biosynthetic pathway. Preferably, the inhibitor is an inhibitor of spermidine biosynthesis. Most preferably, the inhibitor is a spermidine synthase or MTA inhibitor. Particular inhibitors to be comprised in the compositions of the invention are those listed above.

The compositions and methods of the invention are particularly intended for the treatment of OA in humans, but other mammals are also included. These compositions may be administered directly to the subject to be treated or it may be desirable to conjugate them to carrier proteins such as ovalbumin or serum albumin prior to their_administration. Therapeutic formulations may be administered in any conventional dosage formulation. Formulations typically comprise at least one active ingredient, as defined above, together with one or more acceptable carriers thereof.

According to a preferred embodiment, the composition of the invention may optionally further comprise a pharmaceutically or veterinarily acceptable carrier, excipient and/or diluent.

Pharmacology and Drug Delivery

The compounds or pharmaceutical compositions of the present invention are administered and dosed in accordance with good medical practice, taking into account the clinical condition of the individual patient, the disease to be treated, the site and method of administration, scheduling of administration, patient age, sex, body weight and other factors known to medical practitioners.

The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

The treatment generally has a length proportional to the length of the disease process and drug effectiveness and the patient species being treated. It is noted that humans are treated generally longer than the mice or other experimental animals exemplified herein.

The compounds of the present invention can be administered by any of the conventional routes of administration. It should be noted that the compound can be administered as the compound or as pharmaceutically acceptable salt and can be administered alone or as an active ingredient in combination with pharmaceutically acceptable carriers, solvents, diluents, excipients, adjuvants and vehicles. The compounds can be administered orally, subcutaneously or parenterally including intravenous, intraarterial, intramuscular, intraperitoneally, and intranasal administration as well as intrathecal and infusion techniques. Implants of the compounds are also useful. Liquid forms may be prepared for injection, the term including subcutaneous, transdermal, intravenous, intramuscular, intrathecal, and other parental routes of administration. The liquid compositions include aqueous solutions, with and without organic cosolvents, aqueous or oil suspensions, emulsions with edible oils, as well as similar pharmaceutical vehicles. In addition, under certain circumstances the compositions for use in the novel treatments of the present invention may be formed as aerosols, for intranasal and like administration. The patient being treated is a warm-blooded animal and, in particular, mammals including man. The pharmaceutically acceptable carriers, solvents, diluents, excipients, adjuvants and vehicles as well as implant carriers generally refer to inert, non-toxic solid or liquid fillers, diluents or encapsulating material not reacting with the active ingredients of the invention.

When administering the compound of the present invention parenterally, it is generally formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, can also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it is desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

A pharmacological formulation of the compound utilized in the present invention can be administered orally to the patient. Conventional methods such as administering the compound in tablets, suspensions, solutions, emulsions, capsules, powders, syrups and the like are usable. Known techniques which deliver it orally or intravenously and retain the biological activity are preferred. In one embodiment, the compound of the present invention can be administered initially by intravenous injection to bring blood levels to a suitable level. The patient's levels are then maintained by an oral dosage form, although other forms of administration, dependent upon the patient's condition and as indicated above, can be used.

In general, the active dose of compound for humans is in the range of from 1 ng/kg to about 20-100 mg/kg body weight per day, preferably about 0.01 mg to about 2-10 mg/kg body weight per day, in a regimen of one dose per day or twice or three or more times per day for a period of 1-2 weeks or longer, preferably for 24- to 48 hrs or by continuous infusion during a period of 1-2 weeks or longer. Treatment for many years or even lifetime treatment is also envisaged for some of the indications disclosed herein.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

Throughout this application, various publications, including United States patents, are referenced by author and year and patents by number. The disclosures of these publications and patents and patent applications in their entireties are hereby incorporated by reference into this application in order to more fully describe the state of the art to which this invention pertains.

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

Standard molecular biology protocols known in the art not specifically described herein are generally followed essentially as in Sambrook et al., Molecular cloning: A laboratory manual, Cold Springs Harbor Laboratory, New-York (1989, 1992), and in Ausubel et al., Current Protocols in Molecular Biology, John Wiley and Sons, Baltimore, Md. (1988).

Standard organic synthesis protocols known in the art not specifically described herein are generally followed essentially as in Organic syntheses: Vol. 1-79, editors vary, J. Wiley, New York, (1941-2003); Gewert et al., Organic synthesis workbook, Wiley-VCH, Weinheim (2000); Smith & March, Advanced Organic Chemistry, Wiley-Interscience; 5th edition (2001).

Standard medicinal chemistry methods known in the art not specifically described herein are generally followed essentially as in the series “Comprehensive Medicinal Chemistry”, by various authors and editors, published by Pergamon Press.

The following Examples illustrate the present invention in further detail, but the present invention is not limited to these Examples.

### Example 1

An activated hapten, 6-[5-(2,5-dioxopyrrolidin-1-yloxy)pentyl]-5′-deoxy-5′-methylthioadenosine (compound 8) was synthesized according to the following synthesis scheme.
Synthesis Scheme 1

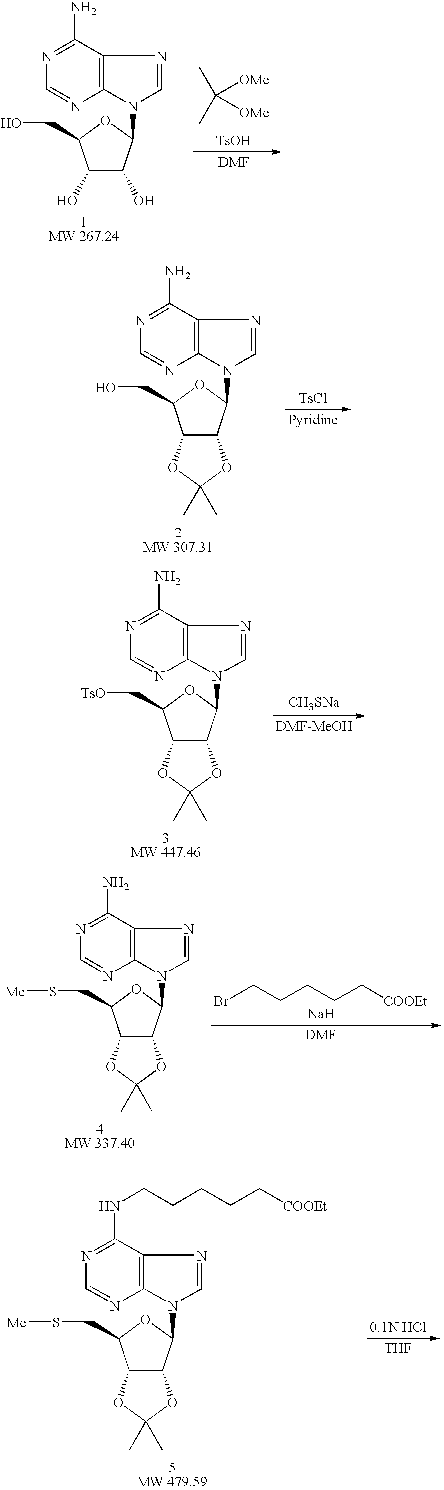

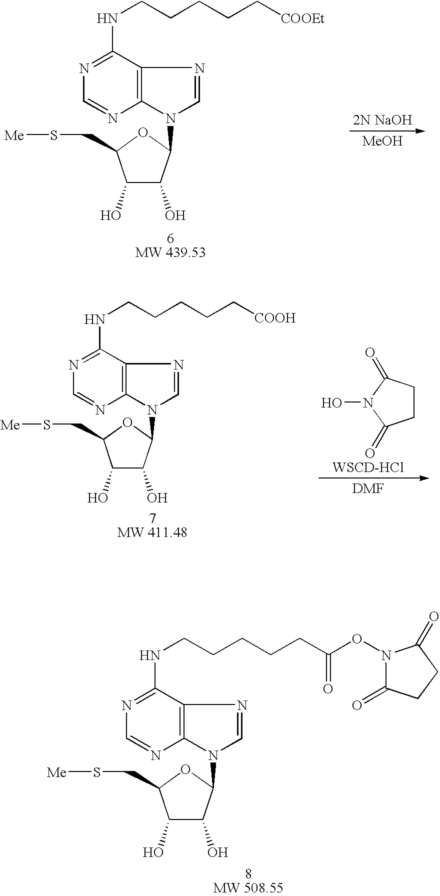

### Preparation of 2′,3′-isopropylideneadenosine

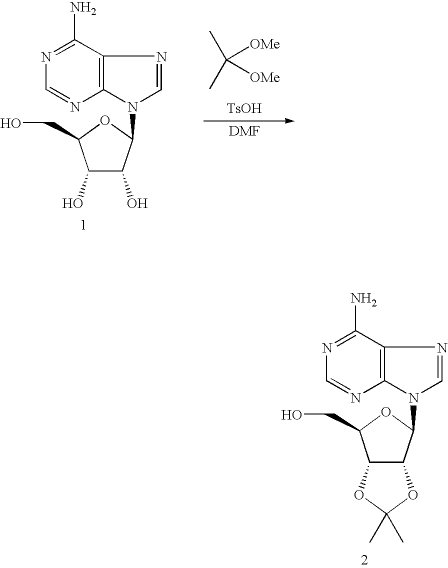

To a solution of 9β-D-ribofaranosyladenosine (adenosine) (1) (6.68 g, 25 mmol) in anhydrous dimethylformamide (100 ml) was added 2,2-dimethoxypropane (13 g, 125 mmol, 5 mol eq.) and anhydrous p-toluenesulfonic acid (1.19 g, 6.26 mmol). The reaction mixture was heated to 70° C. for 7 hours with stirring in a nitrogen stream, and then concentrated in vacuo at 40° C. to about 30 ml. The concentrate was poured into 5% aqueous sodium hydrogen carbonate (125 ml), and extracted twice with 150 ml of ethyl acetate. The organic layer was washed twice with 50 ml of water and 50 ml of saturated aqueous sodium chloride, dried over anhydrous sodium sulfate, and concentrated in vacuo to remove the solvent. The resultant yellow oily residue (ca. 8 g) was dissolved in 150 ml of ethyl acetate with heating. The solution was stirred for 5 hours at room temperature and another 30 minutes at 0° C. to result in precipitation of crystals. The crystals were collected by filtration, and washed with ethyl acetate and diethyl ether to give compound 2 (4.1 g, 13.3 mmol, 53%).

IR (Nujol, cm⁻¹) 3350 (sh), 3240, 3180, 1685 (s).

¹H NMR (200 mHz, CDCl₃) δ: 1.38 (3H, s), 1.65 (3H, s), 3.72-3.86 (1H, m), 3.95-4.02 (1H, m), 4.55 (1H, 8), 5.10-5.25 (2H, m), 5.86 (3H, d, J=4.8 Hz), 6.57 (1H, dd, J=1.8, 11.4 Hz), 7.84 (1H, s), 8.32 (1H, s).

### Preparation of 2′,3′-isopropylidene-5′-(p-toluenesulfonyl)adenosine

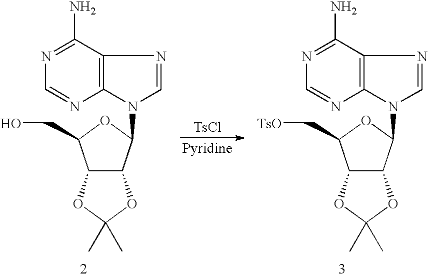

To a solution of 2′,3′-isopropylideneadenosine (2) (4.09, 13 mmol) in anhydrous pyridine (40 ml) was added dropwise a solution of p-toluenesulfonyl chloride (2.97 g, ca. 15.6 mmol, 1.2 mol eq.) in anhydrous pyridine (6 ml) at −20° C. in a nitrogen stream, and the resultant mixture was allowed to stand for 4 days in the dark. For treatment of unreacted reagents, water (4.0 ml) was added to the reaction mixture at −20° C., and the mixture was stirred for 15 minutes. After further addition of water (200 ml), the mixture was extracted twice with 80 ml of ethyl acetate. The organic layer was washed with 50 ml of water, 80 ml of 5% sulfuric acid twice and then 50 ml of saturated aqueous sodium chloride thrice, dried over anhydrous sodium sulfate, and concentrated in vacuo to remove the solvent. The resultant yellow oily residue was applied on a column chromatography (Merck silicagel No. 9385, 230-400 mesh: 50 g) and eluted with ethyl acetate to give a crystalline residue (ca. 6 g), which was washed with diethyl ether to give compound 3 (4.49 g, 9.75 mmol, 75%).

¹H NMR (200 mHz, CDCl₃+D₂O) δ: 1.37 (3H, s), 1.59 (3H, s), 2.40 (3H, s), 4.20-4.36 (2H, m), 4.44-4.53 (1H, m), 5.05 (1H, dd, J=3.4, 6.2 Hz), 5.34 (1H, dd, J=1.8, 6.2 Hz), 6.05 (1H, d, J=18 Hz), 7.19 (2H, d, J=8.4 Hz), 7.63 (2H, d, J=8.4 Hz), 7.84 (1H, s), 8.23 (1H, s).

### Preparation of 2′,3′-isopropylidene-5′-deoxy-5′-methylthioadenosine

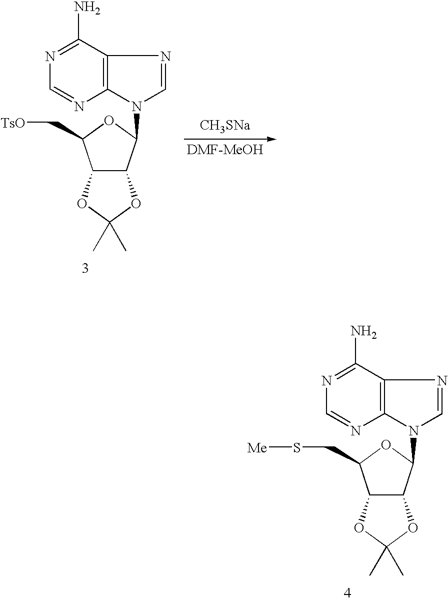

To a solution of sodium thiomethoxide (5.0 g, 71.4 mmol, 8.24 mol eq.) in dimethylformamide-methanol (2:1, 35 ml) was added 2′,3′-isopropylidene-5′-(p-toluenesulfonyl)adenosine (3) (4 g, 8.67 mmol) at 0° C. in a nitrogen stream, and the resultant mixture was allowed to stand for 3 days at 5° C. in the dark. The reaction mixture was concentrated in vacuo to about 20 ml at not higher than 30° C. The concentrate was poured into 250 ml of cold water and extracted twice with 100 ml of ethyl acetate. The organic layer was washed with 50 ml of cold water thrice and 50 ml of saturated aqueous sodium chloride twice, dried over anhydrous sodium sulfate, and concentrated in vacuo to remove the solvent. The resultant crystalline residue was recrystallized from ethyl acetate to give compound 4 (2.8 g, ca. 8.3 mmol, 95%).

¹H NMR (200 mHz, CDCl₃) δ: 1.41 (3H, s), 1.62 (3H, s), 2.11 (3H, s), 267-2.91 (2H, m), 4.36-4.48 (1H, m), 5.07 (1H, dd, J=2.2, 3.4 Hz), 5.52 (1H, dd, J=2.2, 6.2 Hz), 5.64 (2H, br), 6.09 (1H, d, J=2.2 Hz), 7.93 (1H, s), 8.36 (1H, s).

### Preparation of 6-(5-ethoxycarbonylpentyl)-2′,3′-isopropylidene-5′-deoxy-5′-methylthioadenosine

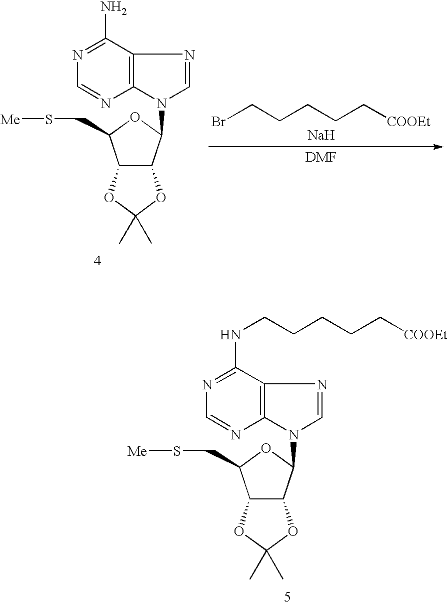

To a solution of 2′,3′-isopropylidene-5′-deoxy-5′-methylthioadenosine (4) (1.50 g, 4.45 mmol) in anhydrous dimethylformamide (10 ml) was added sodium hydride (112 mg, 4.67 mmol) portionwise at room temperature in a nitrogen stream, and then stirred for 2 hours. Then, to the mixture was added dropwise 6-bromoethyl hexanoate (1.04 g, 4.67 mmol) in anhydrous dimethylformamide (3 ml). The reaction mixture was stirred for 1 hour at room temperature and for another 2 hours at 60° C., and then poured into 60 ml of cold water and extracted twice with 40 ml of ethyl acetate. The organic layer was washed with 30 ml of cold water thrice and 30 ml of saturated aqueous sodium chloride twice, dried over anhydrous sodium sulfate, and concentrated in vacuo to remove the solvent. The resultant oily residue was subjected to column chromatography (Merck silica gel, Lobar column size B) and eluted with n-hexane-ethyl acetate (1:1) to give a colorless oily compound 5 (1.4 g, 2.92 mmol, 62.5%).

¹H NMR (200 mHz, CDCl₃) δ: 1.25 (3H, t, J=7.0 Hz), 1.40 (3H, s), 1.45-1.53 (2H, m), 1.62 (3H, s), 1.64-1.79 (4H, m), 2.10 (3H, s), 2.31 (2H, t, J=7.2 Hz), 2.68-2.90 (2H, m), 3.56-3.76 (2H, m), 4.09 (2H, q, J=7.0 Hz), 4.36-4.45 (1H, m), 5.08 (1H, dd, J=3.2, 6.6 Hz), 5.53 (1H, dd, J=1.8, 6.2 Hz), 5.86 (1H, br), 6.07 (1H, d, J=1.8 Hz), 7.86 (1H, s), 8.36 (1H, s).

### Preparation of 6-(5-ethoxycarbonylpentyl)-5′-deoxy-5′-methylthioadenosine

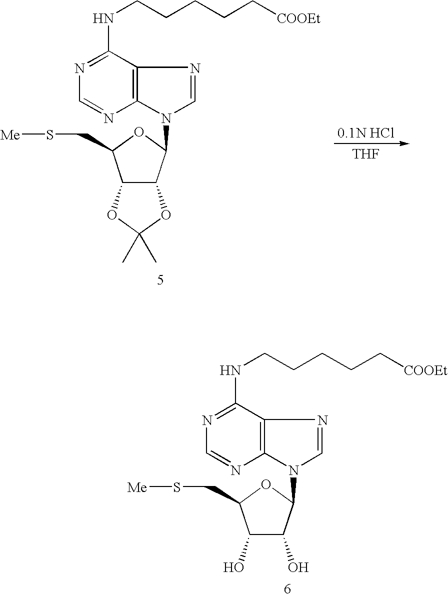

A solution of 6-(5-ethoxycarbonylpentyl)-2′,3′-isopropylidene-5′-deoxy-5′-methylthioadenosine (5) (700 mg, 1.46 mmol), 0.1 N hydrochloric acid (5 ml) and tetrahydrofuran (10 ml) was reacted at 50° C. until no reactant was detected on TLC. The reaction solution was concentrated in vacuo to about 1 ml at 50° C., 5% aqueous sodium hydrogen carbonate (10 ml) was added thereto, and then extracted with 20 ml of ethyl acetate. The organic layer was washed with 10 ml of water twice and 5 ml of saturated aqueous sodium chloride, dried over anhydrous sodium sulfate, and concentrated in vacuo to remove the solvent. The resultant residue was subjected to column chromatography (Merck silica gel, Lobar column size A×2) and eluted with ethyl acetate-acetone (3:1) to give a colorless crystalline compound 6 (250 mg, 0.569 mmol, 39%).

¹H NMR (200 mHz, CDCl₃+D₂O) δ: 1.25 (3H, t, J=7.2 Hz), 1.38-1.56 (2H, m), 1.59-1.81 (4H, m), 2.18 (3H, s), 2.31 (2H, t, J=7.2 Hz), 2.85 (2H, d, J=5.4 Hz), 3.53-3.78 (2H, m), 4.12 (2H, q, J=7.2 Hz), 4.32-4.45 (2H, m), 4.54 (1H, t, J=5.2 Hz), 5.94 (1H, d, J=4.8 Hz), 8.00 (1H, s), 8.29 (1H, s).

### Preparation of 6-(5-carboxylpentyl)-5′-deoxy-5′-methylthioadenosine

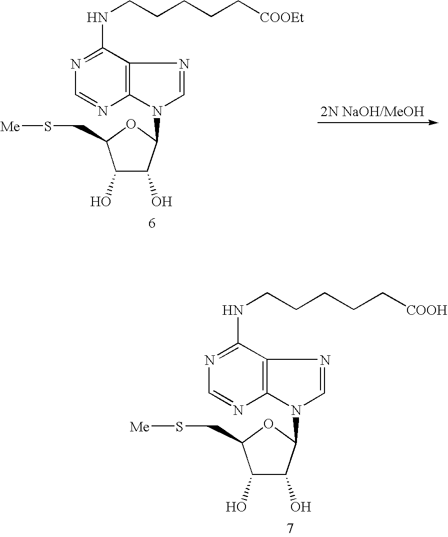

A solution of 6-(5-ethoxycarbonylpentyl)-5′-deoxy-5′-methylthioadenosine (6) (250 mg, 0.569 mmol), 2N sodium hydroxide (1 ml) and methanol (5 ml) was stirred for 1 hour at 60° C. under a nitrogen atmosphere. The reaction solution was concentrated in vacuo to about 1 ml at not higher than 30° C., and diluted with 3 ml of water, and then acetic acid (240 mg, 4 mmol) was added thereto until the aqueous solution became acidic (pH 3). The precipitated crystals were collected by filtration to give compound 7 (223 mg, 0.541 mmol, 95%).

¹H NMR (200 mHz, DMSO-d6) δ: 1.23-1.41 (2H, m), 1.44-1.68 (4H, m), 2.06 (3H, s), 2.20 (2H, t, J=7.2 Hz), 2.47-2.58 (2H, m), 2.72-2.94 (2H, m), 3.98-4.08 (1H, m), 4.13 (1H, t, J=5.4 Hz), 4.75 (1H, t, J=5.4 Hz), 5.50 (3H, br), 5.90 (1H, d, J=1.8 Hz), 7.82 (1H, br), 8.21 (1H, s), 8.86 (1H, s).

### Preparation of 6-[5-(2,5-dioxopyrrolidin-1-yloxy)pentyl]-5′-deoxy-5′-methylthioadenosine

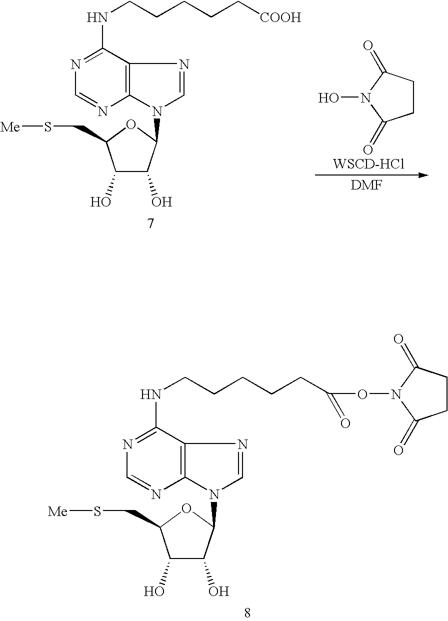

To a solution of N-hydroxysuccinimide (125 mg, 1.08 mmol) and 1-ethyl-3-(3-dimethylaminopropyl)-carbodiimide hydrochloride (WSCD.HCl, 207 mg, 1.08 mmol) in anhydrous dimethylformamide (2 ml) was added a solution of 6-(5-carboxylpentyl)-5′-deoxy-5′-methylthioadenosine (7) (223 mg, 0.541 mmol) in anhydrous dimethylformamide (2 ml) dropwise over 30 minutes at room temperature under a nitrogen atmosphere, and then stirred for 3 hours and half at 40° C. The reaction mixture was poured into 25 ml of cold water and extracted twice with 25 ml of ethyl acetate. The organic layer was washed with 10 ml of cold water twice and 5 ml of saturated aqueous sodium chloride, dried over anhydrous sodium sulfate, and concentrated in vacuo to remove the solvent. The resultant oily residue was subjected to column chromatography (Merck silica gel, Lobar column size A×2) and eluted with ethyl acetate-acetone (3:1) to give a colorless oily compound 8 (231 mg, 0.455 mmol, 80%).

¹H NMR (200 mHz, CDCl₃) δ: 1.50-1.64 (2H, m), 1.68-1.90 (4H, m), 2.18 (3H, s), 2.63 (2H, t, J=57.2 Hz), 2.77-2.89 (6H, m), 3.47 (1H, br), 3.68 (2H, br), 4.35-4.51 (2H, m), 4.57 (1H, t, J=5.4 Hz), 5.91 (1H, d, J=6.0 Hz), 6.01 (1H, br), 6.46 (1H, br), 7.97 (1H, s), 8.29 (1H, s).

### Example 2

### Synthesis of 6-(3-amino)propyl-5′-deoxy-5′-methylthioadenosine

Synthesis Scheme 2

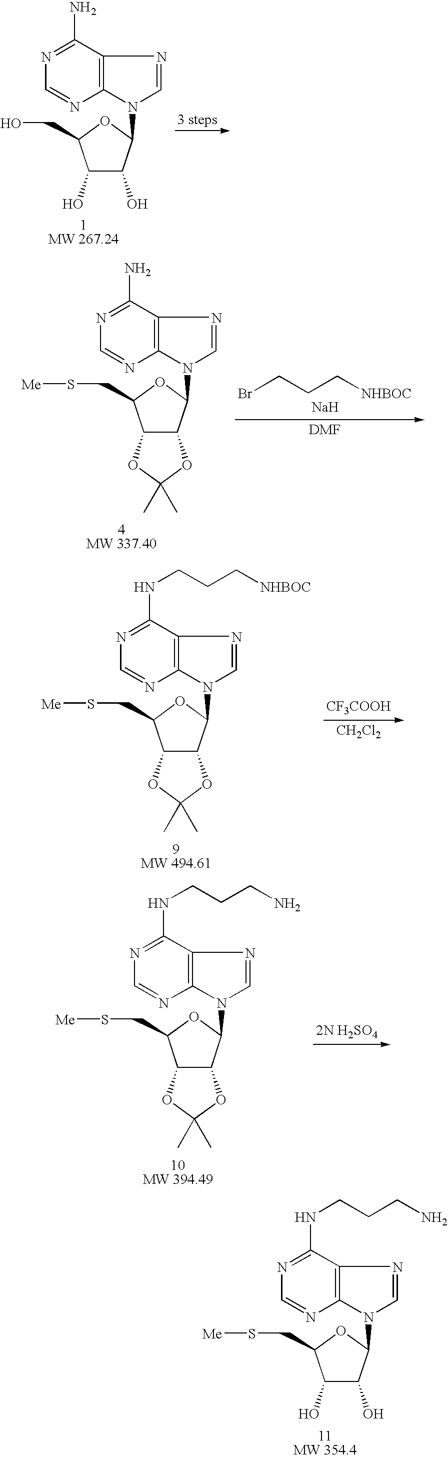

### Preparation of 6-(3-tert-butoxycarbonylamino)propyl-2′,3′-isopropylidene-5′-deoxy-5′-methylthioadenosine

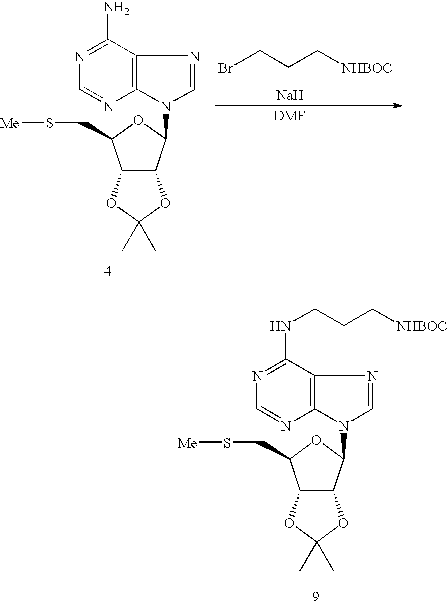

To a solution of 2′,3′-isopropylidene-5′-deoxy-5′-methylthioadenosine (4) (338 mg, 1 mmol) in anhydrous dimethylformamide (2 ml) was added sodium hydride (31 mg, 1.28 mmol) portionwise at room temperature under a nitrogen atmosphere, and the mixture was stirred for 2 hours. Then, to the reaction mixture was added dropwise a solution of 3-(tert-butoxycarbonylamino)propyl bromide (310 mg, 1.3 mmol) in anhydrous dimethylformamide (0.3 ml), and the mixture was stirred for 1 hour at room temperature and for additional 2 hours at 60° C. The reaction mixture was then poured into 20 ml of cold water and extracted twice with 20 ml of ethyl acetate. The organic layer was washed with 10 ml of cold water thrice and 10 ml of saturated aqueous sodium chloride, dried over anhydrous sodium sulfate, and concentrated in vacuo to remove the solvent. The resultant oily residue was subjected to column chromatography (Merck silica gel, Lobar column size A) and eluted with n-hexane-ethyl acetate (2:3) to give a colorless oily compound 9 (242 mg, 0.5 mmol, 50%).

¹H NMR (200 mHz, CDCl₃) δ: 1.40 (3H, s), 1.46 (9H, s), 1.62 (3H, s), 1.75-1.90 (2H, m), 2.11 (3H, s), 2.67-2.91 (2H, m), 3.22 (2H, dd, J=6.0, 12.2 Hz), 3.73 (2H, br), 4.35-4.46 (1H, m), 5.08 (1H, dd, J=3.0, 6.2 Hz), 5.26 (1H, br), 5.52 (1H, dd, J=2.2, 6.2 Hz), 6.07 (1H, d, J=2.2 Hz), 6.18 (1H, br), 7.87 (1H, s), 8.36 (1H, s).

### Preparation of 6-(3-amino)propyl-2′,3′-isopropylidene-5′-deoxy-5′-methylthioadenosine

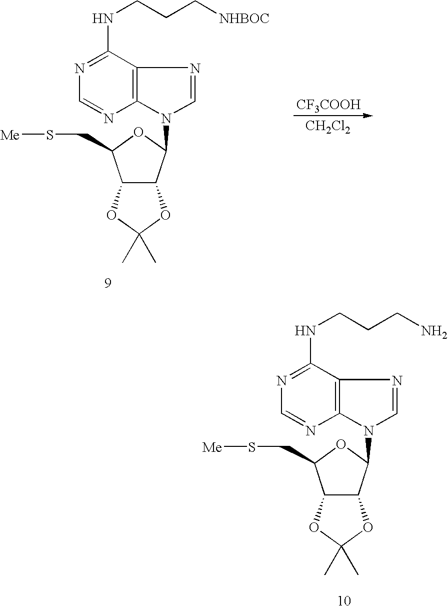

A solution of 6-(3-tert-butoxycarbonylamino)propyl-2′, 3′-isopropylidene-5′-deoxy-5′-methylthioadenosine (9) (242 mg, 0.5 mmol), trifluoroacetic acid (0.5 ml) and dichloromethane (2.5 ml) was reacted at 40° C. until no reactant was detected on TLC. The reaction solution was concentrated in vacuo at not higher than 25° C., and saturated aqueous potassium carbonate (10 ml) was added thereto, and then the mixture was extracted with 20 ml of ethyl acetate. The organic layer was washed with 5 ml of water twice and 5 ml of saturated aqueous sodium chloride, dried over anhydrous sodium sulfate, and concentrated in vacuo to remove the solvent. The resultant residue was subjected to column chromatography (Merck silica gel, thin layer plates KGF 254; 0.5 mm, 20 cm×20 cm, development and elution solvent; ethyl acetate-acetic acid-water=3:1:1) to give a colorless powder compound 10 (180 mg, 0.47 mmol, 94%).

### Preparation of 6-(3-amino)propyl-5′-deoxy-5′-methylthioadenosine

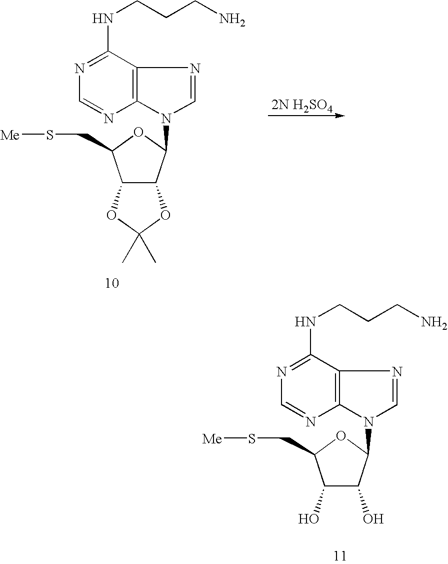

A solution of 6-(3-amino)propyl-2′,3′-isopropylidene-5′-deoxy-5′-methylthioadenosine (10) (180 mg, 0.47 mmol) in 2N sulfuric acid (5 ml) was allowed to stand overnight at room temperature. To the reaction solution was added 5% aqueous potassium carbonate to adjust pH 9, and then the solution was concentrated in vacuo to give a crystalline residue. The residue was extracted with 20 ml of anhydrous ethanol. The extract was concentrated in vacuo to remove the solvent. The resultant extraction residue (140 mg) was applied to preparative thin-layer chromatography (Merck silica gel, thin layer plates KGF 254; 0.5 mm, 20 cm×20 cm, development and elution solvent; ethyl acetate-acetic acid-water=3:1:1) and to HP-20 column chromatography (Mitsubishi Kasei; 75-150 μm, 5 g) and eluted with ethanol-water-28% ammonia water (10:9:1) to give a colorless oily compound 11 (ca. 40 mg).

¹H NMR (200 mHz, CD₃OD) δ: 1.80-1.95 (2H, m), 2.12 (3H, s), 2.80 (2H, t, J=7.0 Hz), 2.91 (2H, t, J=6.0 Hz), 3.70 (2H, br), 4.28 (1H, dd, J=5.8, 10.2 Hz), 4.33 (1H, t, J=4.8 Hz), 4.68-5.00 (6H, m), 6.00 (1H, d, J=5.2 Hz), 8.26 (1H, s), 8.28 (1H, s).

### Example 3

### Labeling of MTA with Horseradish Peroxidase (HRP)

0.63 mg (1.8 mmol) of 6-(3-amino)propyl-5′-deoxy-5′-methylthioadenosine prepared according to Example 2 was dissolved in 0.05 ml of distilled water and 0.1 ml of 0.1M phosphate buffer (pH 7.4). To the solution was added 0.2 ml of an aqueous solution containing 1.19 mg (2.7 μmol) of sulfosuccinimidyl 4-(N-maleimidomethyl)-cyclohexane-1-carboxylate (sulfo-SMCC, Pierce), and reacted for 2 hours at room temperature. By performing a reverse phase HPLC (column: YMC-Pack ODS-A, 6.O I.D.×150 mm, eluting conditions: acetonitrile/0.1% trifluoroacetic acid, 0 to 50%, 20 min, linear gradient, flow rate: 1 ml/min, detection wavelength: 260 nm), a maleimide-introduced MTA fraction (14.7 min) was collected, concentrated in vacuo to remove the solvent, and then the residue was dissolved in 0.4 ml of 20% methanol/distilled water. On the other hand, to 1.1 mg (27 nmol) of horseradish peroxidase (HRP, Roche) in 0.2 ml of 0.1 M phosphate buffer (pH 7.4) was added 0.05 ml of an aqueous solution of 0.43 mg (810 nmol) of sulfosuccinimidyl 6-(3′-[2-pyridyldithio)-propionamido]hexanoate (sulfo-LC-SPDP, Pierce), and the mixture was reacted for 2 hours at room temperature. After addition of 0.05 ml of 0.1 M mercaptoethylamine, the mixture was allowed to stand for 1 hour at room temperature, and then passed through PD-10 (Amersham Pharmacia Biotech) equilibrated with 0.1 M phosphate buffer (pH 6.0) containing 5 mM ethylenediaminetetraacetic acid to remove low molecular weight materials. To 0.2 ml of a fraction containing 0.115 μg (2.9 nmol) of HRP was added 28 μg (49 nmol) of the maleimide-introduced MTA, and the mixture was reacted for 3 days at 4° C. The reaction solution was passed through PD-10 (Amersham Pharmacia Biotech) equilibrated with 0.1 M phosphate buffer (pH 7.4) to remove low molecular weight materials to give HRP-labeled MTA.

### Example 4

### Preparation of a Monoclonal Antibody Recognizing MTA and a Hybridoma Producing the Antibody

(1) Preparation of an Immunogen and Immunization

Keyhole limpet hemocyanin (KLH, Pierce) was used as a carrier protein. 2 ml of distilled water was added into a vial with 20 mg of KLH to make a solution of phosphate buffered saline (pH 7.2). To the solution was added 0.19 ml of DMF containing 3.8 mg (7.5 μmol) of 6-[5-(2,5-dioxopyrrolidin-1-yloxy)pentyl]-5′-deoxy-5′-methylthioadenosine prepared according to Example 1, and the mixture was reacted for 2 hours at room temperature to prepare a conjugate. The reaction solution was dialyzed against distilled water at 4° C., and then freeze-dried to give 18.4 mg of conjugate in dry weight. It was used as an immunogen.

1.3 mg of the immunogen was dissolved in 0.5 ml of saline, and 0.8 ml of Freund's complete adjuvant (Difco) was added thereto to make an emulsion. A/J jms Slc mouse (6 weeks of age, female) was immunized by intraperitoneal administration of 0.1 ml (0.1 mg) of the emulsion five times at three-week intervals. The mouse was subjected to cell fusion on the third day after booster intraperitoneal injection of 0.1 ml of saline containing 0.1 mg of the immunogen.

(2) Preparation of a Hybridoma Producing an Antibody and MTA-7H8 Antibody by Using the Hybridoma

On the third day after the last administration, an immunized mouse according to the procedure in the above (1) was extracted to collect spleen cells. 1.0×10⁸ of the spleen cells were fused with 2×10⁷ of mouse myeloma cells (p3×63-Ag8.UI, Tokyo Cancer Institute) using 50% polyethylene glycol 4000, and selection was performed on a medium containing hypoxanthine, aminopterin and thymidine. On the tenth day of the cell fusion, screening for specific antibody-producing cells was performed. ELISA used for the screening was as follows: 1 μg of anti-mouse IgG antibody (Shibayagi) in 100 μl of PBS (phosphate buffer containing 0.15 M NaCl (pH 7.4)) was added to each well of a 96-well microtiter plate (Nunc), and immobilized for 16 hours at 4° C. These wells were washed with 200 μl of PBS once, and then added with 200 μl of BlockACE (Dainippon Pharmaceutical Co., Ltd.) and allowed to stand for 16 hours at 4° C. to perform blocking (anti-mouse IgG antibody-immobilized plate). Each well was washed with 300 μl of buffer A (50 mM Tris buffer containing 0.01% Tween 20, 0.05% Proclin150 and 0.15 M NaCl (pH 7.4)) once, and then 50 μl of buffer B (50 mM Tris buffer containing 0.5% bovine serum albumin, 0.01% Tween20, 0.05% Proclin150 and 0.15 M NaCl (pH 7.4)), and 50 μl of hybridoma culture supernatant were added thereto, and the mixture was reacted for 3 hours at room temperature. Then, each well was washed thrice with 300 μl of buffer A, and added with 100 μl of HRP-labeled MTA made to 20 ng/ml with buffer B, and allowed to react for 16 hours at 4° C. Each well was washed again, and then added with 100 μl of TMB+Substrate-Chromogen (Dako) and allowed to color for 30 minutes at room temperature. Then, each well was added with 100 μl of 1N sulfuric acid to stop the reaction, and absorbance of each well was measured at 450 nm by using 1420 ARVO SX multi-label counter (PerkinElmer Life Science).

A hybridoma in a well in which production of specific antibodies was positive was cloned twice by limiting dilution to obtain a clone of hybridoma producing a monoclonal antibody which recognizes MTA.

With respect to the culture supernatant of hybridoma, a subclass of the antibody produced by the hybridoma was determined by using mouse monoclonal antibody isotyping ELISA kit (ED Bioscience). The new monoclonal antibody obtained was named MTA-7H8, and its isotype was IgG1 (k).

The hybridoma producing the monoclonal antibody MTA-7H8 of the present invention was deposited at International Patent Organism Depositary in National Institute of Advanced Industrial Science and Technology (Tsukuba Central 6, 1-1-1 Higashi, Tsukuba, Ibaraki, Japan) on Mar. 2, 2004, under the accession number FERMP-p19707 under the Budapest Treaty, and subsequently deposited at the International patent organism depository (IPOD) in Japan under accession number FERM-ABP10226.

Antibody Titer of Murine Antiserum

A 1,000-fold to 1,000,0000-fold dilution series of=antiserum was prepared by repeating a procedure of diluting 10-fold an antiserum collected from the immunized mouse with buffer B. The antimouse IgG antibody-immobilized plate was washed once with 300 μl of buffer A, and then added with 100 μl of diluted antiserum, and allowed to react for 3 hours at room temperature. Then, each well was washed with 300 μl of buffer A thrice, and then added with 100 μl of HRP-labeled MTA made to 20 ng/ml with buffer B, and allowed to react overnight at 4° C. Then, each well was washed with 300 μl of buffer A thrice, and then added with 100 μl of TMB+Substrate-Chromogen (Dako) and allowed to color for 30 minutes at room temperature. Then, each well was added with 100 μl of 1N sulfuric acid to stop the reaction, and absorbance at 450 μm was measured by using 1420 ARVO SX multi-label counter (PerkinElmer Life Science). The dilution rate of the antiserum in which the absorbance is 1.0 was set to an antibody titer. The measurement results showed that the polyclonal antibody (antiserum) of the present invention has the antibody titer of 1,000,000-fold, which is 500 times larger than that of the prior art.

Reactivity of Anti-MTA Antibody to MTA and dcSAM

A 10-fold dilution series of 0.2 to 200 pmol/ml for MTA and a 10-fold dilution series of 2 to 2,000 pmol/ml for dcSAM were prepared with buffer B. Each well of the antimouse IgG antibody-immobilized plate was washed once with 300 μl of buffer A, and then added with 100 μl of MTA-7H8 made to 10 ng/ml with buffer B or 100 μl of murine antiserum diluted 1,000,000-fold, and allowed to react for 3 hours at room temperature. Then, each well was washed with 300 μl of buffer A thrice, and then added with 50 μl of a diluted solution of MTA or dcSAM and 50 μl of HRP-labeled MTA made to 40 ng/ml with buffer B, and allowed to react overnight at 4° C. Then, each well was washed with 300 μl of buffer A thrice, and then added with 100 μl of TMB+Substrate-Chromogen (Dako) and allowed to color for 30 minutes at room temperature. Then, 100 μl of 1 N sulfuric acid was added to each well to stop the reaction, and absorbance at 450 nm was measured by using 1420 ARVO SX multi-label counter (PerkinElmer Life Science).

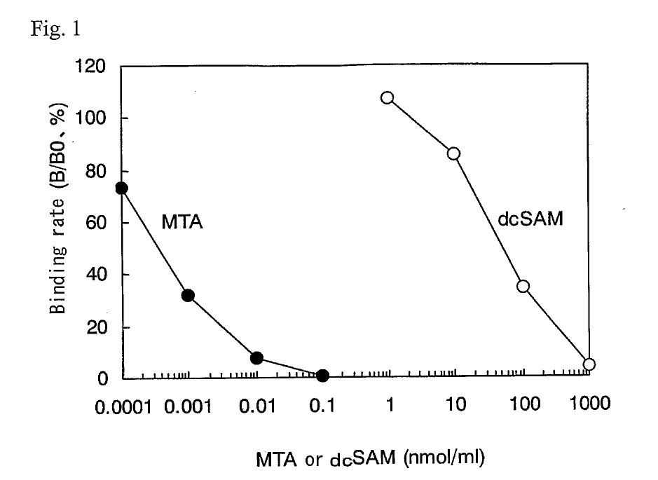

The result of reactivity of MTA-7H8 was shown in FIG. 1. Competitive curves were produced by plotting a calculated ratio (B value/B₀ value (%)) of an absorbance at a concentration (B value) to an absorbance at zero-concentration (B₀ value) in ordinate versus a concentration of MTA or dcSAM in abscissa. In FIG. 1, open circles represent the reactivity to dcSAM and filled circles represent the reactivity to MTA. Affinities (IC50 values) of MTA-7H8 to MTA and dcSAM in the competitive curves were 0.4 nM and 50 μM, respectively. As described above, it was confirmed that the present antibody can detect MTA specifically, because MTA-7H8 not only shows high reactivity to MTA, but also has very small cross-reactivity of only 0.0008% to dcSAM which is a precursor of MTA. Moreover, affinities (IC50 values) of the polyclonal antibody (antiserum) of the present invention to MTA and dcSAM in the competitive curves were 4 nM and 300 μM, respectively, and the cross-reactivity was 0.001%. Therefore, the polyclonal antibody (antiserum) of the present invention was greatly superior to a polyclonal antibody (1%) of the prior art. The results are listed together with the prior art (J. Biochem. Mol. Biol., 1997, Vol. 30, p403) in Table 1.

TABLE 1

| | | | Affinity | | Cross- |
|---|---|---|---|---|---|
| | | Antibody | (IC50 | | reactivity |
| Example | Type | titer | value) | | (dcSAM) |
| Example 4 | Monoclonal | | 0.4 | nM | 0.0008% |
| Example 4 | Polyclonal | 1,000,000 | 4 | nM | 0.001% |
| J. Biochem. Mol. | Polyclonal | 2,000 | 10 | nM | 1% |
| Biol., 1997, | | | | | |
| Vol. 30, p 403 | | | | | |

### Example 5

### Purification of Monoclonal Antibody MTA-7H8

1×10⁷ of hybridomas obtained in Example 4 per Balb/c mouse were provided to Laboproducts Inc. to outsource preparation of ascites. The obtained ascites was centrifuged at 10,000×g for 20 minutes. The supernatant thereof was diluted two-fold with PBS, and passed through Protein G-Sepharose column (Amersham Pharmacia Biotech). After adequately washing the column with PBS, antibodies were eluted with 0.2 M glycine hydrochloride buffer (pH 2.7), and added immediately with 1 M Tris buffer to neutralize. An equal amount of saturated ammonium sulfate was added to the solution, and the mixture was allowed to stand for 30 minutes at 4° C. to salt out. The materials salted out were collected by centrifuging at 10,000×g for 20 minutes. The resultant precipitate was dissolved in PBS, and then dialyzed against PBS at 4° C. overnight to give a purified monoclonal antibody.

### Example 6

### Purification of Murine Anti-MTA Polyclonal Antibody

To 1.5 ml of coupling gel in AminoLink immobilizing kit (Pierce) was added 0.63 mg (1.8 μmol) of 6-(3-amino)propyl-5′-deoxy-5′-methylthioadenosine prepared according to Example 2 in 1.5 ml of 0.1 M phosphate buffer (pH 7.4), and a column containing MTA immobilizing gel was prepared according to the procedure manual of the kit. To the column, which was pre-equilibrated with 0.1 M phosphate buffer (pH 7.4), was added a serum of the above-described immunized mouse diluted 3-fold with 0.1 m phosphate buffer (pH 7.4). After sufficiently washing the column with the same buffer, an antibody was eluted with 6 mM hydrochloric acid, dialyzed against 0.1 M phosphate buffer (pH 8.0) at 4° C. to give a purified polyclonal antibody.

### Example 7

### Preparation of Recombinant Human Spermidine Synthase

An expression vector was constructed by using Gateway Technology of Invitrogen. Human SPDS gene was amplified by PCR, inserted into an entry vector pENTR11 via SacI and NotI restriction enzyme sites to give an entry clone. A gene linked to 3×FLAG tag (Sigma) and Reading Frame Cassette A of Gateway Vector Conversion System was amplified by PCR, inserted into pShuttle vector (Clontech) via NheI and KpnI restriction enzyme sites to give a destination vector. By mixing the entry clone and the destination vector in the presence of a Clonase to induce a site-specific recombination between att sites, an expression vector of human SPDS linked to 3×FLAG tag at N-end was cloned. The expression vector was purified by using EndFree Plasmid Maxi kit (Qiagen).

HEK293 cells (derived from human embryonic kidney, available from the American Type Culture Collection) were transfected by using Lipofectamine 2000 (Invitrogen) and the expression vector according to the procedure manual. The cells were cultivated for 3 days, harvested, suspended into a small amount of TBS (50 mM Tris buffer (pH 7.4) containing 0.15 M NaCl), and then sonicated. A solution of sonicated cells was passed through anti-FLAG antibody (M2)-agarose column (Sigma) equilibrated with TBS. The column was adequately washed with TBS, and then washed with TBS containing 0.1 mg/ml of 3×FLAG peptide (Sigma). An eluant was collected by 1 ml each. An activity of each fraction was measured by HTRF with 1 μl of the fraction, and fractions having an activity were mixed, added with 1 mM DTT, 10% glycerol and 0.05 mM dcSAM, and then stored at −80° C. By western blotting with anti-MTA antibody, purity of a purified SPDS was confirmed.

### Example 8

### Activity Measurement of Spermidine Synthase by ELISA

The recombinant human spermidine synthase was diluted with buffer C (0.1 M potassium phosphate buffer (pH 7.4) containing 0.1% bovine serum albumin), and 50 μl of the solution was added to a 96-well assay plate (Corning). To the plate was added 50 μl of substrate mixture (0.1 M potassium phosphate buffer (pH 7.4) containing 0.4 mM dithiothreitol, 0.1 mM dcSAM, 1.3 mM putrescine, 0.4 mM adenine and 0.1% bovine serum albumin), and the mixture was allowed to react at room temperature. Then, to each well was added 100 μl of buffer C containing 20 mM 4-methylcyclohexylamine (Tokyo Kasei Kogyo) to stop the reaction. 10 μl of the reaction solution was transferred into the microtiter plate to which the above-described anti-mouse IgG antibody was immobilized, and added with 40 μl of HRP-labeled MTA made to 50 ng/ml with buffer B and 50 μl of MTA-7H8 monoclonal antibody made to 40 ng/ml with buffer B, and the mixture was allowed to react at room temperature overnight. Then, each well was washed with 300 μl of buffer A thrice, added with 0.1 ml of TMB+Substrate-Chromogen (Dako), and the mixture was allowed to react to color for 30 minutes at room temperature. Then, each well was added with 100 μl of 1N sulfuric acid to stop the reaction, and absorbance at 450 nm was measured by using 1420 ARVO SX multi-label counter (PerkinElmer Life Science). The amount of MTA in each well was calculated from the competitive curve described above, and the produced amount of MTA per an enzyme amount and a reaction time was determined to be defined as an enzyme activity.

Results

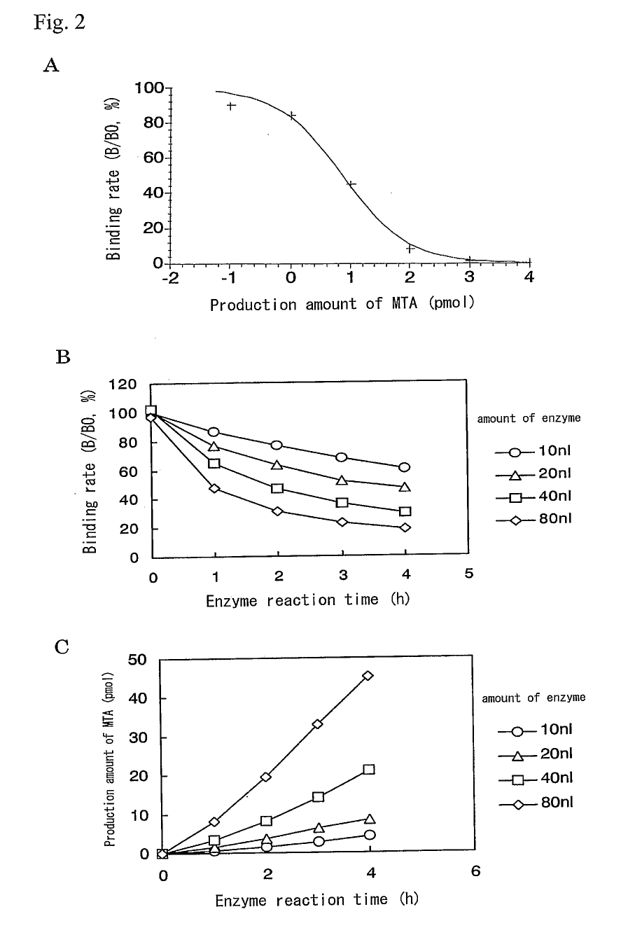

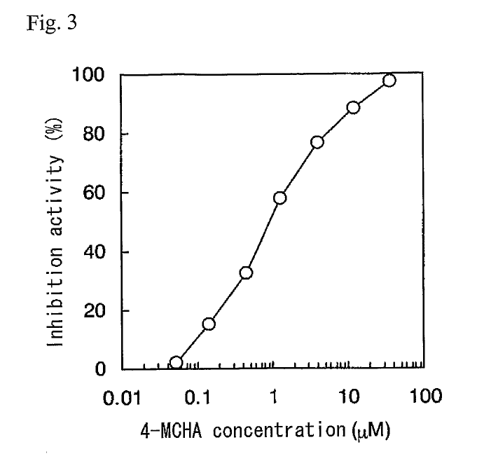

The result of activity measurement of spermidine synthase by ELISA is shown in FIG. 2. It was confirmed that a production amount of MTA was increased depending on a concentration of spermidine synthase and a reaction time. The result of activity measurement in the case of adding 4-methylcyclohexylamine which is a known inhibitor of spermidine synthase in an enzymatic reaction is shown in FIG. 3. Depending on a concentration of 4-methylcyclohexylamine, production of MTA was inhibited, and IC50 value was 1.0 μM similar to a value, 1.7 μM in a publication (Shirahata et al., Biochem. Pharma., 1991.). In other words, it was confirmed that the present method could measure an activity of spermidine synthase, as MTA detected by the present method reflected a result of conversion of putrescine to spermidine by spermidine synthase. When searching an inhibitor of spermidine synthase, 1 μl of a test substance dissolved in 10% DMSO was previously added to a well in the plate, and the above-described reaction was conducted. By detecting a production amount of MTA according to the present method, an inhibition effect of the test substance was measured.

### Example 9

### Activity Measurement of Spermidine Synthase by HTRF

(1) Labeling New Monoclonal Antibody MTA-7H8 with Europium Cryptate

690 μg of MTA-7H8 monoclonal antibody (4.6 nmol) was dissolved in 770 μl of 0.1 M phosphate buffer (pH 8.0) and added with 100 μg of europium TBP monosuberate (CIS Bio International) (68 nmol), and then the mixture was reacted for 1 hour at room temperature. The reaction solution was passed through PD-10 (Amersham Pharmacia Biotech) equilibrated with PBS to remove low molecular weigh materials to give europium cryptate-labeled MTA-7H8 monoclonal antibody.

(2) Labeling MTA with a Cross-Linked Allophycocyanin (XL665)

7.7 mg of XL665 (CIS Bio International) (74 nmol) was dissolved in 1.1 ml of 0.1 M phosphate buffer (pH 7.0) and added with 140 μg of N-Succinimidyl 3-(2-pyridyldithio)propionate (SPDP, Pierce) (443 nmol). The mixture was allowed to react for 20 minutes at room temperature, and further added with dithiothreitol to a final concentration 10 mM, and then reacted for 10 minutes to introduce a cysteine group into XL665. The reaction solution was passed through PD-10 (Amersham Pharmacia Biotech) equilibrated with 0.1 M phosphate buffer (pH 7.0) containing 10 mM EDTA to remove low molecular weigh materials to give a fraction containing XL665. 1.3 ml of the fraction containing 3.4 mg (33 nmol) of XL665 was added with 660 nmol of a solution of the maleimide-introduced MTA, and the solution was reacted at 4° C. overnight. To the reaction solution was added 300 μg of N-ethylmaleimide (2.4 μmol), and the mixture was allowed to stand for 10 minutes at room temperature, and then passed through PD-10 (Amersham Pharmacia Biotech) equilibrated with 0.1 M potassium phosphate buffer (pH 7.4) to remove low molecular weigh materials to give XL665-labeled MTA.

(3) Measurement of Spermidine Synthase Activity

10 μL of spermidine synthase diluted with buffer C was added into 384-well assay plate (black, Corning). Then, to the plate was added 10 μl of the above substrate mixture, and reaction was conducted at room temperature. Each well was added with 20 μl of buffer D (0.5% bovine serum albumin and 0.6 M potassium fluoride in 0.1 M potassium phosphate buffer, pH 7.4) containing 30 mM 4-methylcyclohexylamine and 260 ng/ml XL665-labeled MTA to stop the reaction. Then, each well was added with 20 μl of europium cryptate-labeled anti-MTA-7H8 antibody made to 288 ng/ml with buffer D, and the mixture was reacted for 16 hours at room temperature. Fluorescence of each well was measured by Rubyster (BMG Labtechnologis). An amount of MTA in each well was calculated from a standard curve, and converted to an amount of MTA per an enzyme amount and a reaction time, which was an enzyme activity.

Results

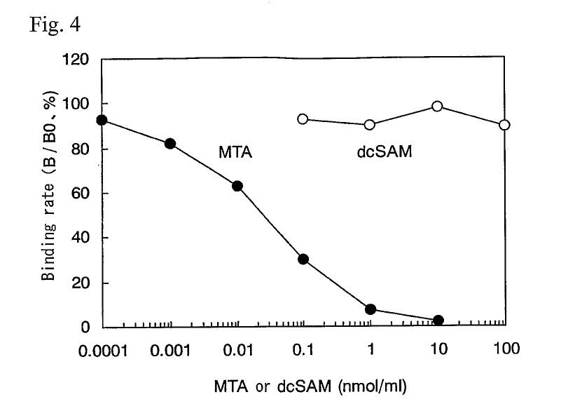

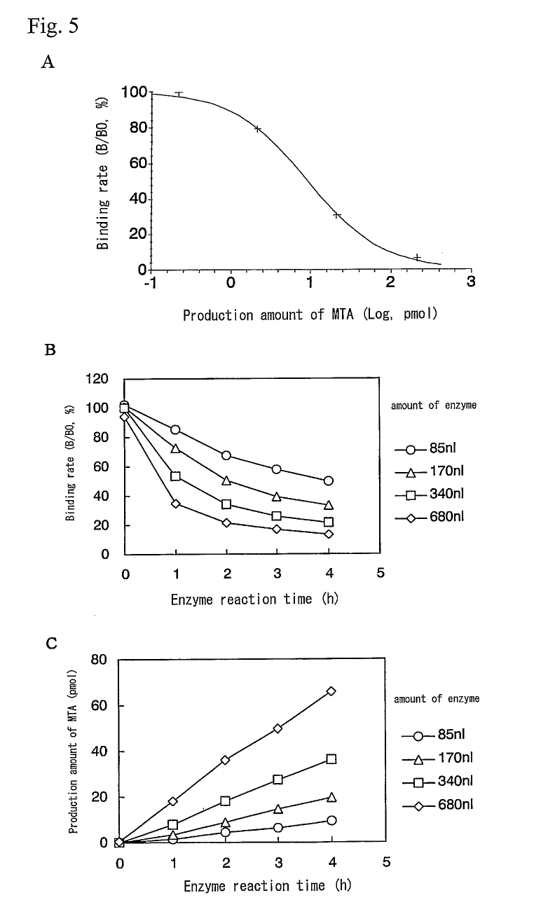

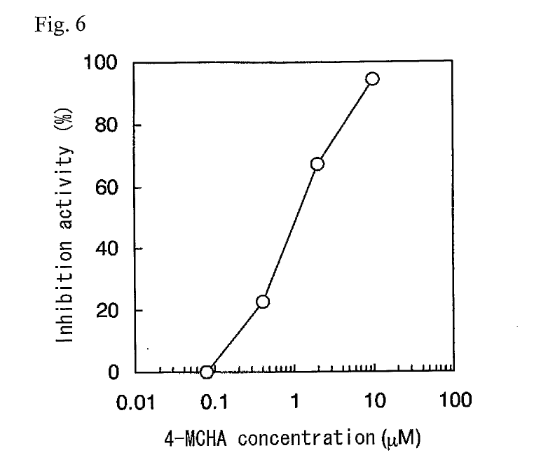

A standard curve in HTRF is shown in FIG. 4. In FIG. 4, open circles represent reactivity to dcSAM and filled circles represent reactivity to MTA. Although MTA is inferior in the sensitivity compared to ELISA, it maintains a high sensitivity as 20 nM of IC50 value and has very small cross-reactivity of only 0.02% to the substrate dcSAM, and therefore an activity of spermidine synthase could be measured by the present method. The measurement result of activity spermidine synthase activity by HTRF is shown in FIG. 5. It was confirmed that a production amount of MTA was increased depending on a concentration of spermidine synthase and a reaction time. The result of activity measurement in the case of adding 4-methylcyclohexylamine which is a known inhibitor in an enzymatic reaction is shown in FIG. 6. Production of MTA was inhibited depending on a concentration of 4-methylcyclohexylamine, and IC50 value was 1.1 μM, which was similar to the result of ELISA. In other words, it was confirmed that the present method could measure an activity of spermidine synthase, as MTA detected by the present method reflected a result of conversion of putrescine to spermidine by spermidine synthase. In the case of screening an inhibitor of spermidine synthase, 1 μl of a test substance dissolved in 10% DMSO was previously added to a well in the plate, and the above-described reaction was conducted. By detecting a production amount of MTA according to the present method, an inhibition effect of the test substance was measured.

### Example 10

### Quantification of MTA Using SPA

(1) Labeling MTA with ¹²⁵I

The solvent in 9×10⁶Bq radioiodine [¹²⁵I]-Bolton-Hunter reagent (PerkinElmer Life Science) was removed in vacuo, and to this were added 10 μl of 20% aqueous methanol and 30 μl of 25 mmol/L phosphate buffer (pH 7.1) containing 13 μg of 6-(3-amino)propyl-5′-deoxy-5′-methylthioadenosine prepared according to Example 2 and then the solution was reacted at 4° C. overnight. The reaction solution was subjected to a reverse phase high performance liquid chromatography (instrument: LC-6A manufactured by Shimadzu, column: YMC-Pack ODS-A 6.0 I.D.×150 mm (YMC), eluent A: 10% acetonitrile/0.1% trifluoroacetic acid, eluent B: 60% acetonitrile/0.1% trifluoroacetic acid, A→B, linear gradient (25 min), flow rate: 1 ml/min), and ¹²⁵I-labeled MTA (about 2.6×10⁶ Bq) eluting at 16 minutes was collected.

(2) Quantification of MTA

To a microtiter plate (IsoPlate-96, white, PerkinElmer Life Science) were added sequentially 50 μl of SPA PVT beads linked to anti-mouse antibodies (Amersham Pharmacia Biotech) made to 20 mg/ml with measurement buffer E (0.5% bovine serum albumin in 0.1 M potassium phosphate buffer (pH 7.4)), 50 μl of MTA standard solution, 50 μl of ¹²⁵I-labeled MTA made to 12,000 Bq/ml with measurement buffer E, and finally 50 μl of measurement buffer E containing 1,000 ng/ml of mouse polyclonal antibody, and the mixture was reacted at room temperature overnight. After that, each well was measured for radioactivity by a microplate scintillation counter (Top Count NXT, PerkinElmer Life Science) to give a standard curve.

Results

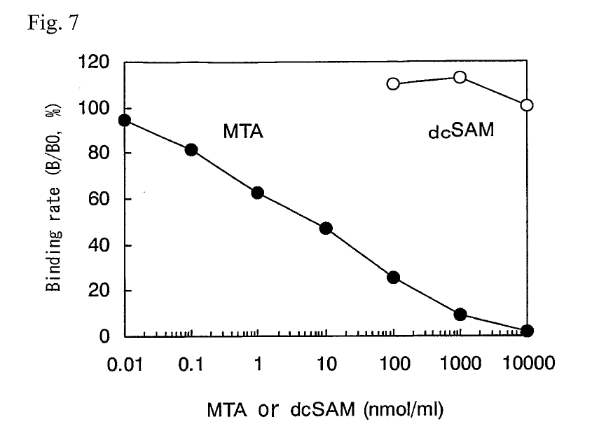

The standard curve obtained by SPA is shown in FIG. 7. Although MTA was inferior compared to ELISA and HTRF described above, it had 10 μM of IC50 value and a very small cross-reactivity of only 0.1% to dcSAM. Therefore, it was confirmed that MTA could be quantified by the present method. By using the present method, an activity of spermidine synthase can be measured. In the case of screening an inhibitor of spermidine synthase, 1 μl of a test substance dissolved in 10% DMSO was previously added to a well in the plate, and the above-described reaction was conducted. By detecting a production amount of MTA according to the present method, an inhibition effect of the test substance was measured.

### Example 11

### Quantitative Determination of Spermidine by HPLC

The published method of Saeki et al. (J. Chromatogr. 1978. 221-229.) was modified for this study. SPDS reactions (40 μL each) were performed as described above and terminated by addition of 10 μL of 50 mM HCl instead of 4-MCHA. To these reaction mixtures 10 μL of 10% Trichloroacetic acid was added, and the mixtures were centrifuged. To 20 μL of supernatants and spermidine standard solutions 20 μL of 500 mM Na2CO3 solution and 40 μL of 20 mM dansyl chloride dissolved in acetonitrile were added, and these mixtures were incubated in 40° C. for 30 min. After the incubation, the mixtures were centrifuged and 20 μL of supernatants were used for measuring spermidine. Dansylated spermidine was separated and quantitated by reversed-phase HPLC (Column: Cadenza CD-C18, 4.6 mm I.D.×75 mm, Mobile phase A: 50 mM ammonium formate (pH5.0)/acetonitrile (90:10, v/v), Mobile phase B: 50 mM ammonium formate (pH5.0)/acetonitrile (10:90, v/v), Gradient elution: from 55% B (0 to 3 min) to 100% B (3 to 10 min), Flow rate 1 ml/min, Detection: excitation 360 nm, emission 510 nm).

Results

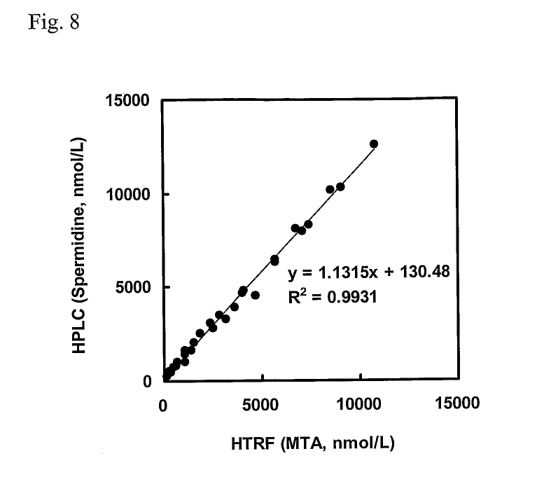

The present invention measures MTA but not spermidine, which is a major product of the SPDS reaction. When spermidine is produced by SPDS, the same number of MTA molecules is produced. To investigate whether SPDS activity could be specifically measured by the present invention or not, the amount of spermidine produced by SPDS was determined using HPLC and the result was compared with that of the present invention. Polyamines in SPDS reactions were dansylated and then dansylated spermidine was measured by HPLC. In the HPLC method, retention time of dansylated spermidine was around 13 min. Correlation between MTA levels determined by the present invention and spermidine levels measured by HPLC method is shown in FIG. 8. MTA levels (x) correlated well with spermidine levels (y) (y=1.1315x+130.48 nmol/L, R2=0.9931, n=28). This result demonstrates that the present invention can specifically measure SPDS activity.

1-33. (canceled)

34. A monoclonal antibody which binds to an immunogen comprising a 5′-deoxy-5′-methylthioadenosine moiety, wherein the antibody is specifically directed to such moiety.

35. The monoclonal antibody according to claim 34 having less than 0.1% of cross-reactivity to an immunogen comprising a decarboxylated S-adenosylmethionine, based on the reactivity of the antibody to 5′-deoxy-5′-methylthioadenosine being taken as 100%.

36. The monoclonal antibody according to claim 35, wherein the cross-reactivity is less than 0.01%.

37. The monoclonal antibody according to claim 34, which is raised against an immunogen selected from the group consisting of:
(a) a carrier protein covalently bound to 5′-deoxy-5′-methylthioadenosine, wherein the bond is between the protein and the purine group of 5′-deoxy-5′-methylthioadenosine;
(b) an immunogen according to (a) represented by the general formula: 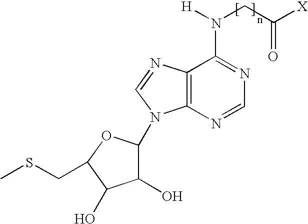
wherein n is an integer greater than or equal to 1, and X is the carrier protein; and
(c) the immunogen of (b) wherein n equals 5.

38. A composition comprising a monoclonal antibody from claim 34 and a detectable label attached thereto.

39. The composition according to claim 38, wherein the label is an enzyme, a fluorescent substance, a colored bead, a radioisotope, a metal or biotin.

40. The composition according to claim 39, wherein the label is peroxidase.

41. The composition according to claim 39, wherein the label is europium cryptate.

42. A method for treating a subject in need of treatment for a disease involving spermidine synthase which comprises administering to the subject an amount of an antibody which inhibits spermidine biosynthesis sufficient to effect substantially complete inhibition of spermidine biosynthesis so as to thereby treat the subject.

43. The method of claim 42, wherein the disease is a proliferative disease selected from the group consisting of cancer, osteoporosis, Alzheimer's disease, and hypertension.

44. The method of claim 42, wherein the disease is osteoarthritis.

45. The method of claim 42, wherein the antibody binds to an immunogen comprising a 5′-deoxy-5′-methylthioadenosine moiety, wherein the antibody is specifically directed to such moiety.

46. A composition comprising the antibody of claim 34, and a carrier.

47. The composition of claim 46, wherein the carrier is a pharmaceutically acceptable carrier.

48. A hybridoma which produces a monoclonal antibody which binds to an immunogen comprising a 5′-deoxy-5′-methylthioadenosine moiety, wherein the antibody is specifically directed to such moiety.

49. A hybridoma according to claim 48 deposited under accession number FERM-ABP10226.

50. A process of preparing the antibody which binds to an immunogen comprising a 5′-deoxy-5′-methylthioadenosine moiety, wherein the antibody is specifically directed to such moiety, using the hybridoma of claim 48.

51. A process according to claim 50 comprising cultivating the hybridoma and purifying the culture supernatant.
